(19) United States
(12) Patent Application Publication
(10) Pub. No.: US 2026/0227396 A1
(43) Pub. Date: Aug. 6, 2026

(54) METHOD FOR IDENTIFYING A MULTI-PARAMETER PHENOTYPE OF MICROBIOTA

(71) Applicant: Deutsches Rheuma-Forschungszentrum Berlin, Berlin (DE)

(72) Inventors: Hyun-Dong CHANG, Berlin (DE); Lisa BUDZINSKI, Berlin (DE)

(21) Appl. No.: 19/150,512

(22) PCT Filed: Jan. 24, 2024
(86) PCT No.: PCT/EP2024/051617

§ 371 Date: Jul. 23, 2025

(30) Foreign Application Priority Data
Jan. 24, 2023 (EP) 23153069.2

(51) Int. Cl. G01N 33/569; G01N 15/10; G01N 15/1429; G01N 33/58; G16H 10/40; G16H 50/20

(52) U.S. Cl. CPC G01N 33/56911; G01N 15/1429; G01N 33/58; G16H 10/40; G16H 50/20; G01N 2015/1006; G01N 2469/20

(57) ABSTRACT

A method for identifying a multi-parameter phenotype of microbiota. The method includes (i) providing a sample including microbiota, (ii) labeling the microbiota with multiple labels, each of which binds a phenotypic parameter of the microbiota, (iii) detecting an intensity of the labelled phenotypic parameters of single cells of the microbiota by flow cytometry, and (iv) segmenting the single cells into bins based on the intensities of detected phenotypic parameters, wherein the distribution of single cells in bins represents a multi-parameter phenotype of said microbiota. Also described is a system for identifying a multi-parameter phenotype of intestinal microbiota, a kit for identifying a multi-parameter phenotype of intestinal microbiota and methods for diagnosing a medical condition associated with microbiota, for example an inflammatory condition, such as an inflammatory bowel disease, in a subject.

### DESCRIPTION

The present invention is in the field of flow cytometry and microbiota analysis, and methods for characterizing microbiota, such as those associated with medical conditions, for example inflammatory conditions, including inflammatory bowel disease (IBD), IgG4-related diseases or rheumatoid arthritis.

One aspect of the invention relates to a method for identifying a multi-parameter phenotype of microbiota, comprising:
i) Providing a sample comprising microbiota,
ii) Labeling said microbiota with multiple labels, each of which binds a phenotypic parameter of said microbiota,
iii) Detecting an intensity of the labelled phenotypic parameters of single cells of the microbiota by flow cytometry, and
iv) Segmenting the single cells into bins based on the intensities of detected phenotypic parameters, wherein the distribution of single cells in bins represents a multi-parameter phenotype of said microbiota.

Another aspect of the invention relates to a system for identifying a multi-parameter phenotype of intestinal microbiota, comprising
a) A flow cytometer,
b) A data store comprising one or more reference values, intensity values and/or data defining one or more bins based on phenotypic parameters of single cells of the microbiota, and
c) A software configured for analyzing the intensities of labelled phenotypic parameters of single cells of the microbiota, using data generated by flow cytometry, and segmenting the single cells into bins based on the intensities of detected phenotypic parameters.

A further aspect of the invention relates to a kit for identifying a multi-parameter phenotype of microbiota, preferably intestinal microbiota, comprising
a) Multiple labels, each configured for binding to a phenotypic parameter of microbiota, and
b) A software configured for analyzing the intensities of labelled phenotypic parameters of single cells of the microbiota, using data generated by flow cytometry, and segmenting the single cells into bins based on the intensities of detected phenotypic parameters,
c) wherein said software contains a reference profile for at least one state, preferably two states.

A further aspect of the invention relates to a method for diagnosing a medical condition associated with microbiota, for example an inflammatory condition, such as inflammatory bowel disease, preferably Crohn's disease and/or ulcerative colitis, in a subject, comprising:
i) Identifying a multi-parameter phenotype of intestinal microbiota of a subject with a method as used in the first aspect of the invention, and
ii) Evaluating the phenotype using a machine-learning model that is trained on one or more correlations between a multi-parameter phenotype of said microbiota and one or more states in a subject.

### BACKGROUND OF THE INVENTION

The human intestinal microbiota is intricately linked to human health. It plays an essential role in host energy homeostasis and metabolism, but also contributes significantly to the maturation of the immune system. In consequence, alterations in the composition of the intestinal microbiota have been associated with various human diseases ranging from metabolic or chronic inflammatory diseases, and cancer to neurological disorders [1-4].

Typically, the microbiota composition is characterised by sequencing the highly conserved 16S rRNA gene, encoding a ribosomal RNA, which contains variable regions allowing the determination of phylogenetic relationships between bacteria, and thus taxonomic classification. Applied to cohorts of e.g., patients and healthy controls, 16S rRNA sequencing has revealed alterations in the abundance or presence of certain bacterial taxa, the overall compositional diversity, and the microbial load in multiple diseases. Nevertheless, the high cross-sectional diversity in the microbiota composition in humans has interfered with the identification of systemic and common taxonomic microbial signatures required for disease-classification. Thus, despite the fact that such studies present seminal work for research on the pathogenic potential of certain intestinal microbes, lack of generalization potential and robustness have so far precluded routine clinical application towards the benefit of patients [5-9].

Results from the Human Microbiome Project indicate that despite high taxonomic diversity between individuals, functions of the microbial community are rather conserved, suggesting that the composition of microbial communities is governed by functionality and interaction with the host [10]. Also, in patients with ulcerative colitis (UC) and Crohn's disease (CD), the microbiota has been found to have distinct metabolome profiles compared to healthy donors [11, 12]. Additionally, severe changes in the host-microbiota interaction and modified immune responses towards components of the microbiota are reflected by an altered coating of bacteria by host immunoglobulins [13-15].

Flow cytometry is a widely used tool to rapidly investigate cellular properties on single-cell level, but its potential has not yet been fully explored for microbiota analysis. Microbiota flow cytometry (MFC), has been shown to be an effective method to capture dynamics of microbial communities [16-20], assessing their complexity and compositional changes by light scatter properties and quantitative DNA staining [16-18, 21, 22]. The suitability of MFC for monitoring microbiota dynamics in disease has been demonstrated for murine colitis [19], during chemotherapy-treatment of patients with hematological cancer [20] and to discriminate CD patients from healthy donors [23]. Thus, MFC reflects compositional diversity as seen by 16S rRNA sequencing. However, as 16S rRNA sequencing, MFC does not integrate potentially insightful functional parameters reflecting e.g. host interaction in a disease-specific context.

So far the diagnosis of microbiota-associated disease, or the inspection of environments involving microbiota, e.g., the inspection of wastewater, has focused on analyzing the composition of the microbiota by various sequencing technologies or gene profiling. Such approaches cannot however follow the increasing complexity and rapid compositional changes in microbiota, that may occur in in a human microbiome.

Inflammatory bowel disease (IBD), which is known to be associated with human microbiota, comprises two major disease entities, Crohn's disease (CD) and ulcerative colitis (UC), characterized by chronic, progressive and destructive inflammation of the intestinal tract. While CD can affect the entire intestinal tract from mouth to anus, UC is mainly restricted to the large intestine.

Classical diagnoses of IBD include the assessment of the patient's burden with regard to pain and stool frequency, the analysis of inflammation markers in blood and stool, and inspection of the intestinal tract by ultrasound, resonance tomography or colonoscopy. The most relevant inflammation markers are C-reactive protein (CRP) in serum and fecal calprotectin, but also other fecal markers such as lactoferrin, elastase and lysozyme (ECCO ESGAR guidelines). However, these molecular markers indicate intestinal inflammation as such, but cannot distinguish CD from UC. Thus, a definite discrimination between CD and UC remains a clinical challenge, especially when inflammation persists in the colon.

Multiple studies have aimed at the identification of biomarkers to improve IBD subclassification, such as cytokines or non-coding RNAs, anti-neutrophil serum antibodies, specific microbes or glycans. However, none have found their way into clinical diagnostics today. Hence, the gold standard to discriminate CD and UC remains colonoscopy and the pathological assessment of biopsies (ECCO ESGAR guidelines). Even so, about 10-15% of IBD patients cannot be diagnosed and are termed indeterminate colitis.

The ability to distinguish stool samples of patients with Crohn's disease from healthy controls based on flow cytometric profiles using scatter and DNA staining has been demonstrated in a cohort of 29 CD patients [23]. In another study, a quantitative PCR approach quantifying bacterial and fungal load in stool samples was able to discriminate between CD and UC using a machine-learning algorithm, however, only when also integrating clinical data [29].

In a review article, Özel et al. discuss the importance of dynamic analysis of microbial composition using sequencing and flow cytometry data, emphasizing the potential of real-time quantitative community multiparametric flow cytometry combined with machine learning for rapid and comprehensive microbiome analysis [55]. Although the potential value in diagnostics and treatment of microbiome-related disorders is mentioned, no specific approaches with respect to relevant microbial surface molecules, markers, or clinical conditions are presented.

Kupschus et al. describe the development of an open-source analytic tool for flow cytometry data, allowing statistical analysis and visualization of microbial patterns. The tool comprises comparing results from flow cytometry and 16 S rRNA gene sequencing on human skin samples, demonstrating its ability to discriminate between microbial profiles [22]. The cytometry analysis disclosed therein is based on an analysis using particle size, cell density, and DNA content with the option to incorporate additional information through the use of labeled antibodies.

Rubbens et al. teach an analysis of the gut microbiome in CD patients using flow cytometry and 16 S rRNA analysis, showing that cytometric fingerprints can be used as a diagnostic tool in order to classify samples according to CD state [56]. The cytometry analysis disclosed therein does not involve staining of the microbiome with specific cell surface markers but relies merely on nucleic acid content.

Jackson et al. present an IgA-Seq protocol, with which immunoglobulin binding of commensal bacteria can be assayed by sorting IgG-bound bacteria from samples and using amplicon sequencing to determine taxonomy. The method enables identifying and quantifying commensal gut microbiota targeted by host immunoglobulins [57]. Flow cytometry analysis using IgA staining is disclosed but no mention is made of characterizing microbial populations by multi-parameter cytometry and differentiation between disease states.

In a review article, Bourgonje et al. discuss antibody signatures in inflammatory bowel diseases, such as Crohn's disease and ulcerative colitis, and discuss protein arrays, phage display immunoprecipitation sequencing and B cell receptor sequencing to assess antigens associated with IBD [58]. No mention is made of characterizing microbial populations by multi-parameter cytometry and differentiation between disease states.

While these documents mention the potential use of flow cytometry approaches to characterize microbial populations, they do not provide information relevant microbial surface molecules or markers, or methods of assessing such markers in a multi-parameter approach, to characterize microbial populations. Moreover, the disclosed methods in the prior art do not offer means of reliably distinguishing between related but distinct populations (e.g., different disease states), indicating a limitation in their ability to provide a detailed characterization of closely related microbial populations.

Despite this technology, improved or alternative means for characterizing microbial populations with high accuracy and the capacity for recognizing small but significant differences between microbial populations are required.

Hence, means are required that are capable of identifying complex microbial population characteristics or signatures that correlate with particular states of said population, such as disease states, thus providing improved analytical and diagnostic potential via microbiome analysis.

### SUMMARY OF THE INVENTION

In light of the prior art, the technical problem underlying the present invention is to provide alternative or improved means for characterizing microbial populations.

Another problem underlying the present invention is to provide alternative or improved means for identifying complex microbial population characteristics or signatures that correlate with particular states of said population, such as disease states.

Another problem underlying the present invention is to provide alternative or improved means for classifying a microbial population at sufficient detail to assess the presence or absence, or the differential diagnosis, of a medical condition.

Another problem underlying the present invention is to provide alternative or improved means for identifying or classifying a medical condition, as well as extracting diagnostic parameters, from disease-relevant microbiota features.

Another problem underlying the present invention is to provide alternative or improved means for monitoring or analyzing a certain state of a subject or situation wherein microbiota is involved.

This problem is solved by the features of the independent claims. Preferred embodiments of the present invention are provided by dependent claims.

In a first aspect, the invention therefore relates to a method for identifying a multi-parameter phenotype of microbiota, comprising:
i) Providing a sample comprising microbiota,
ii) Labeling said microbiota with multiple labels, each of which binds a phenotypic parameter of said microbiota,
iii) Detecting an intensity of the labelled phenotypic parameters of single cells of the microbiota by flow cytometry, and
iv) Segmenting the single cells into bins based on the intensities of detected phenotypic parameters, wherein the distribution of single cells in bins represents a multi-parameter phenotype of said microbiota.

As described herein, a method and related aspects are therefore disclosed that enable differentiation between various states of microbial populations, such as disease states, in particular chronic inflammatory diseases, based on a flow cytometric analysis of single microbial cells, thus providing a phenotypic analysis of a bacterial population using multi-parameter flow cytometry.

In embodiments, the present invention thus relates to a multi-parameter flow cytometry approach to analyse single microbial cells, preferably bacterial cells, in complex communities, for example those of the human microbiome (mMFC, multi-parametric microbiota flow cytometry). In embodiments, and as shown in the Examples below, the invention combines the detection of host immunoglobulins (IgA1, IgA2, IgM and IgG) and various surface sugar residues on the bacterial surface. As also shown in the Examples, mMFC is applied to phenotype the microbiota from stool samples of a cohort of patients with inflammatory bowel disease, comprising both CD and UC patients. The inventors applied a self-organizing map (SOM, as in [24]) to identify phenotypically similar cells within the multi-dimensional single-cell data, and performed donor-or cohort-wise statistical evaluations and classification by a random forest machine-learning model. The exemplary approach of the invention was compared to a 16S rRNA gene sequencing-based taxonomic comparison between patient and healthy control samples.

As described in more detail below, the inventors show that mMFC identifies an IBD-specific phenotypic microbiota signature able to robustly classify IBD patients regardless of disease severity, inflammation site, therapy course or IBD-subclass. mMFC is also able to classify CD and UC within the IBD samples. In all instances shown below in the Examples, mMFC classified the samples with higher specificity and sensitivity compared to 16S rRNA gene sequencing-based classification. Furthermore, the inventors show that mMFC identifies IBD-specific microbiota signatures that are distinct from other chronic inflammatory diseases, here rheumatoid arthritis or IgG4-related disease. Altogether, the present invention, employing preferably mMFC-based single-cell phenotyping of intestinal bacteria, represents a novel, fast and robust approach for microbiota analysis suitable for clinical application.

The inventors have further analyzed microbial populations using the method of the invention from subjects with other disease states. Of particular note is that the multi-parameter analysis of microbial populations also allows the identification and characterization of disease states not directly involved in the digestive tract or intestinal health. As shown above, the characterization of disease states is not limited to IBD but enables identification of unique multi-parameter microbial signatures for various inflammatory diseases.

The data disclosed below reveal that clear distinctions are observed between the microbial populations obtained from patients with distinct IBDs, but also in other disease conditions such as Spondyloarthropathy (SpA), rheumatoid arthritis (RA), IgG4-related diseases (IgG4-RD), juvenile idiopathic arthritis (JIA), systemic lupus erythematosus (SLE), and in healthy controls. The method enables means to identify and characterize not only diseases of the digestive tract, but also other medical conditions, for example those with an inflammatory component, and further to non-medical bacterial populations, as disclosed herein.

Also of note is that the present invention enables improvements over earlier nucleic acid analysis, such as rRNA sequence analysis, which is typically used to characterize microbial populations. The present approach therefore provides greater accuracy in distinguishing microbial populations between closely related states at a level of accuracy beyond rRNA techniques presently available.

As described in more detail below, in embodiments, the segmentation and distribution of cells into bins based on their multi-parameter flow cytometric characterization enables the generation of “multi-parameter phenotype” of the microbial population analyzed, and thus provides a “signature” or “characteristic phenotype” capable of defining a microbial population, and optionally comparison between microbial populations to potentially identify differences in any given state between two or more populations.

In embodiments, the method analyzes the surface phenotype of bacteria by assaying for “coating” of intestinal bacteria by host immunoglobulins, preferably of different and multiple antibody-isotypes, and by differentially staining different sugar molecules on the bacterial surface, preferably using plant-derived lectins.

In embodiments, the combined quantitative measurement of the light scatter properties (for example forward and side scatter) of the bacterial cells, and/or assessment of genome size by DNA staining, the invention enables generation of a multi-parametric phenotypic profile of a bacterial population, preferably an intestinal bacterial community of an individual.

In contrast to 16S rRNA gene sequencing, which establishes a unique profile regarding the taxonomic composition of microbiota, the present invention, in embodiments, measures multiple components of potentially functional parameters, i.e. how the host immune system reacts to the microbiota by its antibody response and how the bacteria might react to the intestinal environment by surface sugar expression. Thus, in embodiments, the multi-parameter phenotype of the present invention is composed of not only microbial characteristics but of other markers or components bound to said microbiota, such as antibodies or other agents bound to microbial cells, thereby providing a read-out on microbial-environment interactions. In the case of human microbiota and intestinal microbiomes, the present approach represents an advantageous and unique approach towards characterizing intestinal microbial populations by host factors, such as host antibody response, or other host response that may cause the microbiota to react or present in a particular way.

In embodiments, the multi-dimensional data is then segmented into bins, which categorize the measured bacteria into groups based on phenotypic similarity. Bins, which are significantly over-or underrepresented in a particular state, such as a disease state, can be identified and used in a machine-learning approach. Such approaches allow an automatic or semi-automatic and user-independent distinction between microbial states, either when assessing a single microbial population, or when measured in any two or more populations, such as patients with different diseases and/or healthy controls.

In embodiments, machine learning approaches can be employed in combination with the present invention in order to identify patterns, characteristics and/or signatures of any given multi-parametric phenotypic profile (distribution of cells into bins) and associate these, or the multi-parametric phenotype itself, with any given state, such as a disease state. With sufficient numbers of analyses, a skilled person may establish a machine-learning approach to characterize and associate any given multi-parametric phenotype with any given state, and by training a suitable machine-learning algorithm with a suitable data set, future analyses of a single sample may subsequently be sorted or designated as in a particular state, or determined to exist in a particular state, based on correlations between multi-parametric phenotype and a particular state.

The flow cytometric analysis of stool samples is typically more cost effective, has a much shorter time from sample acquisition to test results (hours instead of days) and requires little to no expert knowledge. Thus, the transferability into a clinical setting and feasibility to use microbiota profiling to support clinical diagnosis is evident.

To the knowledge of the inventors, there has been no suggestion in the art regarding identifying multi-parameter phenotypes of microbiota via a flow cytometric approach, in particular, the combined detection of host immunoglobulin-coating of bacteria, for example by IgA1, IgA2, IgM and IgG, and/or surface sugar residues, detected for example by a combination of plant-derived agglutinins, in order to phenotype intestinal bacteria on the single cell level. This approach in the context of stool samples of CD and UC patients and healthy donors enables a previously unexpected accuracy and automation in determining at times small differences between similar medical conditions. For example, by this approach IBD-specific cytometric microbiota signatures can be identified, which allow the extraction of both diagnostic parameters and disease-relevant microbiota features.

Until now, clinical microbiological analyses are largely limited to the detection of specific pathogens by quantitative PCR and classical microbial cultivation. To the knowledge of the inventors, microbiota-based approaches for clinical diagnostics of non-communicable chronic inflammatory disease are not established.

The ability to distinguish stool samples of patients with Crohn's disease from healthy controls based on flow cytometric profiles using scatter and DNA staining has been demonstrated in a cohort of 29 CD patients (Rubbens et al, ISME J, 2021 January; 15(1):354-358). In another study, a quantitative PCR approach quantifying bacterial and fungal load in stool samples was able to discriminate between CD and UC using a machine-learning algorithm, however, only when also integrating clinical data (Sarrabayrouse et al, mSystems, 2021 Mar, 23;6(2):e01277-20).

The studies described above demonstrate the ability to support diagnosis of inflammatory bowel diseases (IBD) using the relatively simple analysis of the microbiota in stool samples. However, it has not been demonstrated whether the parameters used to predict IBD patients are specific for intestinal inflammation or a general phenomenon of chronic inflammatory diseases, in which a reduction in the complexity of the microbiota is very well established. Due to the low complexity of the data used in the above approaches, it is assumed that distinction between different diseases is not feasible.

In addition, the present approach uses viable bacteria for analysis allowing not only the detection of relevant microbial phenotypes for diagnostics and therapy monitoring/prediction but also the direct isolation of bacteria of interest for downstream analysis regarding their immunological role or pathophysiological impact.

The present invention therefore represents an original approach to analyze multiple (e.g., surface) properties of viable bacterial cells (e.g., isolated from human sample material). In embodiments, the invention comprises a data analysis pipeline that can take the multi-dimensional information and generate an individual microbiota fingerprint or signature. From this microbiota fingerprint or signature, specific features can be extracted, which allow the specific identification of specific states, such as various chronic inflammatory diseases. In embodiments, the extraction of features can be adapted to clinical or research scenarios, e.g. features differentiating between disease and health, different diseases, therapy response, disease prognosis, and others. The present invention thus provides a fast and robust technology for microbiota analyses.

The present invention is preferably characterized by relatively straightforward and simplistic approach, that can be established in existing clinical or analytical settings. Sample staining and measurement as well as the data analysis as such can be standardized and thus translated into clinical application without major obstacles.

Additional fields of application relate to identification of multi-parameter phenotype of microbiota via flow cytometric approach in the inspection of the environment or in industrial settings involving microbiota, such as monitoring wastewater, investigation of vegetation and/or food inspection.

In the context of the present invention, the method is not limited to a particular technical field. Based on the surprising findings of the method disclosed herein, the invention is applicable in any technical field where microbiota play an important role or microbiota need to be inspected, for example, microbiota associated with a medical condition, wastewater surveillance, vegetation monitoring and/or food inspection, etc. The method according to the invention can be applied in any industrial operation or setting where the microbiome is involved. The method here involving characterization of phenotypic parameters of the microbiome can be developed and applied by a skilled person into any necessary format that is useful in an industrial operation or setting, capable of analysis using the method and other means outlined herein.

In embodiments, the invention relates to a method for identifying a multi-parameter phenotype of microbiota in wastewater surveillance, comprising:
i) Providing a water sample comprising microbiota,
ii) Labeling said microbiota with multiple labels, each of which binds a phenotypic parameter of said microbiota,
iii) Detecting an intensity of the labelled phenotypic parameters of single cells of the microbiota by flow cytometry, and
iv) Segmenting the single cells into bins based on the intensities of detected phenotypic parameters, wherein the distribution of single cells in bins represents a multi-parameter phenotype of said microbiota.

Without being bound by the theory, the phenotypic parameters of the wastewater microbiome can be determined by the method as described herein. The determined multi-parameter phenotypes are specific to a state, for example the incidence of an epidemic, suggesting potential association with such a state. Surprisingly, the method as used herein focuses on measuring multi-parameter phenotypes of microbiota and this overcomes a shortcoming of wastewater surveillance, in that current approaches typically focus on a single pathogen at a time. A further advantage is that the method as described herein characterizes a phenotype of microbiota more specifically and accurately than previous approaches, encompassing a more detailed multi-parametric picture of a microbial population, that correlates to a certain state of the microbiota.

In embodiments, a self-organizing map algorithm is applied to analyze multi-dimensional flow cytometry data of phenotypic parameters. Such a self-organizing map is an established technique of the prior art which can make high-dimensional data easier to visualize and analyze. In the context of the invention, the term multi-dimensional refers to at least one, two, three, four, five, six, seven, eight, nine, ten, eleven or twelve dimensions, or more. In embodiments, at least five dimensions of phenotypic parameters are employed. In embodiments, five to eleven dimensions are employed in the method as used herein.

In embodiments, the method as used herein further comprises analyzing the binned cells (multi-parameter phenotype) and identifying correlation(s) between any given sample and a reference phenotype, or between two or more states. In the context of the invention, a state is not limited to a medical condition. A state refers to any condition of a microbiota population, such as a condition of wastewater, condition of water bodies, condition of food, condition of air, condition of vegetation, condition of environment, and the like.

In embodiments, the method as used herein further comprises analyzing the binned cells (multi-parameter phenotype) and identifying correlation(s) between two or more states, wherein the state is a medical condition.

In embodiments, the method comprises analyzing the binned cells (multi-parameter phenotype) and identifying correlation(s) between two or more states, wherein a subject has a medical condition, such as IBD, and the states are for example CD and UC.

In embodiments, the method as used herein is applied for subclassifying human intestinal microbiota, for example, into states of CD and UC. Advantageously, the method according to the invention reveals that the intestinal microbiota possesses a unique and disease-specific phenotype.

It is advantageous that the method as used herein presents the potential of microbiota flow cytometry, apart from cross-validating diversity reduction in a dysbiotic condition, as a diagnostic tool providing information on microbiota complexity, microbiota-host interaction, and microbiota surface composition. Approaches interrogating microbial diversity, for example via nucleic acid sequencing approaches, thus significantly lack in the comparative depth and complexity of the phenotype obtained for any given microbial population.

A further advantage of the invention is that the phenotypes of IBD microbiota more specific than phenotypes of microbiome composition. IBD microbiota are impacted by a particular disease or lifestyle condition of an individual, like immunosuppressive therapeutics or patients' nutrition preferences.

A further advantage is that certain genera of bacteria can be identified by the method as described herein and define a certain disease, for example, CD and UC. In embodiments, such bacteria may serve as biomarkers in defined conditions.

Some bacteria, by themselves, have a relatively high predictive value for disease diagnosis, but are only detected in about 50% of IBD patients (mainly CD). Thus the overall low number of differentiating genera identified for both modeling approaches (IBD vs. HC, CD vs. UC) indicates the extensive impact of donor individuality on the microbiome. This generally makes microbiome-based disease-classification using bacterial identification (via sequencing) less powerful with small cohorts or for individual sampling.

The unique, disease-specific microbiota phenotype as described herein highlights disease individuality, for example, Crohn's disease and ulcerative colitis, which are both associated with a diversity reduction, and further suggests a different role of the intestinal microbiota in each disease pathogenesis. The data disclosed below also reveal that clear distinctions are observed between the microbial populations obtained from patients with distinct IBDs, but also in other disease conditions such as Spondyloarthropathy (SpA), rheumatoid arthritis (RA), IgG4-related diseases (IgG4-RD), juvenile idiopathic arthritis (JIA), systemic lupus erythematosus (SLE), and in healthy controls.

The invention therefore enables insight into various medical conditions, such as those with an inflammatory component, i.e. any inflammatory disease, by multi-parameter analysis of the microbiome obtained from a subject. In embodiments, the invention may therefore be considered a high-resolution sensor for inflammatory status of a subject. In embodiments, by applying the machine-learning approaches disclosed herein, various inflammatory states of subjects may be characterized and thus employed in diagnostics or therapy guidance. The invention therefore enables therapy monitoring and assessment of whether inflammatory conditions are addressed by any given therapy, by measuring, over time, changes in microbial populations and potentially comparing the microbiome of any individual to established patterns or signatures representing particular disease states or therapy responses.

In embodiments, the method further comprises
i) additionally analyzing the binned cells (multi-parameter phenotype) using a predetermined model comprising one or more established patterns (signatures) associated with a state, and/or
ii) additionally analyzing the binned cells (multi-parameter phenotype) using a machine learning model trained on correlation(s) between a phenotype and any given state, or between two or more states.

In the context of the present invention, the predetermined model can attribute the associated state based on the intensities of detected phenotypic parameters of binned cells. In embodiments, flow cytometer data are segmented into bins, wherein each bin comprises phenotypically similar cells. Using a model to identify a pattern or correlation between phenotype and an associated state allows automated analysis based on a machine-learning approach. In embodiments, a suitable machine learning model is trained on one or more correlation(s) between a phenotype and any given state, or between two or more states, such that the subsequent analysis of any given sample may allow matching by way of the trained algorithm to a particular state, for which a correlation has been identified using a suitable training set.

In embodiments, a predetermined model can be generated by using a discriminatory feature, either from a multi-parameter phenotype of the microbiota determined by flow cytometry and/or 16s gene sequencing. In the context of environmental monitoring involving microbiota, for example monitoring the quality of the water bodies, the predefined model is able to give e.g., information on water quality based on the determined phenotype of the microbiota.

It is advantageous that an automatic characterization of a state of a subject is enabled which provides an effective method in classifying disease as well as in determination of any given state in other technical fields.

In embodiments, the state is a medical condition in a subject. In embodiments, the medical condition is associated with microbiota. Medical condition associated with microbiota can be understood as medical condition being caused by microbiota, involving microbiota, or suspect of having microbiota.

In embodiments, the medical condition in a subject is a chronic inflammatory disease.

In embodiments, the phenotypic parameter of the microbiota labelled in step ii) is a structural feature of a microorganism and/or human immunoglobulin bound to the microorganism.

For application of the method as used herein in the medical field, for example in determination of a medical state, the phenotypic parameter as used herein may be a structural feature of the microorganism, in other words, a marker, molecule or other structural component of the microorganism itself. In this context, in embodiments, the phenotypic parameter may preferably be an immunoglobulin of the host bound to the microorganism.

For application of the method in technical fields other than the medical field, a structural feature of a microorganism is a preferred measured parameter.

In embodiments, the label in step ii) comprises a binding agent that specifically recognizes a host immunoglobulin or a sugar molecule present on the surface of a cell of the microbiota.

In the context of the present invention, the binding agent is not limited to recognizing a host immunoglobulin or a sugar molecule present on the surface of a cell of microbiota. Various cell surface markers or structures may be targets of labelling the bacteria. The binding agent may, in embodiments, target any one or more of a glycoprotein, the bacterial genome or parts of the bacterial genome, a structural protein present on the cell surface, or any other factor characterizing for example cell size.

In embodiments, the binding agent that specifically recognizes a host immunoglobulin is an antibody or antigen binding fragment thereof that binds an immunoglobulin isotype IgA1, IgA2, IgG, IgD, IgE and/or IgM.

Advantageously, the host immunoglobulin can reflect the reaction of the host immune system to the microbiota. By exploiting this phenomenon, the present invention enables the phenotyping of microbiota based on its interaction with the host, thus providing additional factors in the analysis beyond simple microbial profiling via sequence data.

In embodiments, the binding agent that specifically recognizes a sugar molecule binds preferably a lactose-, mannose- and/or N-Acetyl-glucosamine-structure present on the surface of a cell of the microbiota.

In embodiments, the label is preferably a plant-derived lectin selected from one or more of peanut agglutinin (PNA), Concanavalin A (ConA), Solanum tuberosum agglutinin (STA) and wheat germ agglutinin (WGA).

In embodiments, step ii) comprises additionally using a label that binds a nucleic acid molecule in single cells of the microbiota, and selecting cells with a positive nucleic acid molecule signal prior to grouping in step iv).

Advantageously, selecting cells with a positive nucleic acid molecule signal can exclude non-cellular debris and instrument noise. Sequence-specific labeling of particular bacterial nucleic acid sequences is envisaged.

Essentially any given structural characteristic of a bacterial cell may be interrogated using the method described herein, preferred embodiments are provided above. Preferably surface accessible markers are employed, as commonly employed in flow cytometric analysis, thus enabling simple detection with established labelling strategies.

With regard to the data processing, in embodiments, each bin is defined by at least one intensity value of a label binding to a host immunoglobulin and at least one intensity value of a label binding to a sugar molecule.

Through this combination of immunoglobulin and sugar detection differentiating multi-parameter phenotypes may be obtained that enable medical conclusions, in particular with respect to IBD. In a preferred embodiment, the invention relates to a method for identifying a multi-parameter phenotype of microbiota, comprising:
i) Providing a sample comprising microbiota,
ii) Labeling said microbiota with multiple labels, each of which binds a phenotypic parameter of said microbiota,
iii) Detecting an intensity of the labelled phenotypic parameters of single cells of the microbiota by flow cytometry, and
iv) Segmenting the single cells into bins based on the intensities of detected phenotypic parameters, wherein the distribution of single cells in bins represents a multi-parameter phenotype of said microbiota,
   wherein the phenotypic parameter of the microbiota labelled in step ii) comprise a human immunoglobulin bound to the microorganism, and a sugar molecule present on the surface of a cell of the microbiota.

In embodiments, the method as used herein comprises selecting one or more discriminatory bins for distinguishing between two inflammatory bowel diseases.

In further preferred embodiments, the method comprises selecting one or more discriminatory bins for distinguishing between any two medical states or conditions, for example, and without limitation, any given IBD, HC, CD, UC, but also in other disease conditions such as Spondyloarthropathy (SpA), rheumatoid arthritis (RA), IgG4-related diseases (IgG4-RD), juvenile idiopathic arthritis (JIA), systemic lupus erythematosus (SLE), and in healthy controls.

Advantageously, discriminatory bins represent discriminatory features between two states, for example between two inflammatory bowel diseases, or between inflammatory bowel disease and the healthy control. Advantageously, based on the identified discriminatory features, one or more predictive models can be generated and tested as to whether the model is able to attribute cohort identity to each sample.

In embodiments, the method as used herein comprises generating a pattern for an inflammatory bowel disease based on the distribution of single cells into bins based on the intensities of detected phenotypic parameters, or based on the discriminatory bins. In embodiments, a similar pattern of microbiota phenotype can attribute a certain state to a subject, such as inflammatory bowel disease. Microbiota patterns may comprises discriminatory features which are able to distinguish one or more states, such as inflammatory bowel disease and healthy controls, or Crohn's disease and Ulcerative colitis.

In embodiments, the sample is a stool sample or saliva, nasopharyngeal swab, skin swab or any sample comprising intestinal microbiota.

In embodiments, the sample can be collected from wastewater, any other body of water, from vegetation, food, air, or from any relevant sample in the environment involving microbes.

In embodiments, the method as used herein comprises training a machine-learning model on one or more correlations between a multi-parametric phenotype and any given state, or between two or more states, wherein the segmenting (or distribution) of single cells into bins based on the intensities of detected phenotypic parameters represents a multi-parametric phenotype. This phenotype and its associations or correlations to any given state, as may be obtained from a study of multiple samples or subjects, may be provided as a training set for a machine-learning model in order to further refine the model and enable automatic characterization of a sample based on established correlations.

In embodiments, the two states are a medical condition, preferably an inflammatory bowel disease, and a healthy condition in a subject.

Another aspect of the invention relates to a system for identifying a multi-parameter phenotype of intestinal microbiota, comprising
a) A flow cytometer,
b) A data store comprising one or more reference values, intensity values and/or data defining one or more bins based on phenotypic parameters of single cells of the microbiota, and
c) A software configured for analyzing the intensities of labelled phenotypic parameters of single cells of the microbiota, using data generated by flow cytometry, and segmenting the single cells into bins based on the intensities of detected phenotypic parameters.

In embodiments, the software is configured for identifying a medical condition, such as an inflammatory bowel disease, associated with a signature for said condition, such as an inflammatory bowel disease, based on the grouping of single cells into bins based on the intensities of detected phenotypic parameters.

A further aspect of the invention relates to a kit for identifying a multi-parameter phenotype of microbiota, preferably intestinal microbiota, comprising
a) Multiple labels, each label configured for binding to a phenotypic parameter of microbiota, and
b) A software configured for analyzing the intensities of labelled phenotypic parameters of single cells of the microbiota, using data generated by flow cytometry, and segmenting the single cells into bins based on the intensities of detected phenotypic parameters,
c) wherein said software contains a reference profile for at least one state, preferably two states.

A further aspect of the invention relates to a method for diagnosing a medical condition associated with microbiota, for example an inflammatory bowel disease, preferably Crohn's disease and/or ulcerative colitis, in a subject, using the method as described herein.

In embodiments, the method comprises:
i) Identifying a multi-parameter phenotype of intestinal microbiota of a subject with a method as described herein, and
ii) Evaluating the phenotype using a machine-learning model that is trained on one or more correlations between a multi-parameter phenotype of said microbiota and one or more states in a subject.

In embodiments, the medical condition associated with microbiota is chronic inflammatory disease preferably selected from the group consisting of inflammatory bowel disease, preferably Crohn's disease and/or ulcerative colitis, rheumatoid arthritis, juvenile arthritis, an IgG4-related disease and/or a kidney disease, such as chronic kidney disease.

The embodiments describing the method of the invention may be used to describe the system of the invention, or the kit, and vice visa. This also applies to any embodiments used to describe the method and the kit, or the system and kit, the method for diagnosis. The invention is unified by the novel and beneficial method for identifying a multi-parameter phenotype of microbiota and thus the relevant features described herein for one aspect may be used to describe any given aspect of the invention, in a manner in conformity with the understanding of a skilled person.

### DETAILED DESCRIPTION OF THE INVENTION

All cited documents of the patent and non-patent literature are hereby incorporated by reference in their entirety.

In embodiments, a method for identifying a multi-parameter phenotype of microbiota, comprising: i) providing a sample comprising microbiota, ii) labeling said microbiota with multiple labels, each of which binds a phenotypic parameter of said microbiota, iii) detecting an intensity of the labelled phenotypic parameters of single cells of the microbiota by flow cytometry, and vi) segmenting the single cells into bins based on the intensities of detected phenotypic parameters, wherein the distribution of single cells in bins represents a multi-parameter phenotype of said microbiota.

As used herein, the term “microbiota” includes, without limitation, bacteria, archaea, protists, fungi and/or viruses present in an ecological community. In embodiments, the microbiota comprises commensal, symbiotic and/or pathogenic microorganisms, as found in and on all multicellular organisms from plants to animals. Microbiota are important for immunologic, hormonal and metabolic homeostasis of their host. Different microbiota may be present on different parts of the body, prefer different nutrient sources, and/or perform different functions. For example, there is an oral microbiota of the mouth, a microbiota of the skin that has many subcategories (the armpits, nose, feet, etc.), and a gut microbiota, among many others.

The term “microbiome” describes either the collective genomes of the microorganisms that reside in an environmental niche, or the microorganisms themselves, including microbial structural elements, such as proteins/peptides, lipids, polysaccharides, nucleic acids and/or mobile genetic elements, or microbial metabolites, such as, signaling molecules, toxins, and (an)organic molecules.

The term “microbiota” and the term “microbiome” can be used interchangeably.

As used herein, the term “phenotype” shall mean an observable characteristic of an individual resulting from its interaction with the environment. The phenotype of microbiota shall mean a set of observable parameters of the microbiota, resulting from its interaction with the environment.

As used herein, a “phenotypic parameter” of the microbiota refers to any detectable characteristic of a microorganism that can be identified or measured using the approach described herein.

Non-limiting examples of a “phenotypic parameter” are structural features of a microorganism, such as a surface protein or sugar, or any detectable component bound to a microorganism, such as an antibody. Further examples are one or more sugar molecules present on the surface of the microbiota, including but not limited to a lactose-, mannose- and/or N-Acetyl-glucosamine-structure present on the surface of a cell of the microbiota. Other phenotypic parameters of the microbiota may comprise one or more host immunoglobulins binding to the surface of the microbiota, including all immunoglobulin isotypes, such as IgA1, IgA2, IgG, IgD, IgE and/or IgM. In the context of the invention, phenotypic parameters may include any given surface molecules, intracellular proteins found on the cell surface, or any surface molecules coated to the surface of the microorganism from the host or the environment.

Additional phenotypic parameters of microbiota include, without limitation, bile salt hydrolase, Dnak, Phosphoglycerate mutase, Ef-Tu, Hsp60, Enolase, Glucose 6-phosphate isomerase, Glutamine synthetase, GAPDH, Alcohol acetaldehyde, Peroxisomal catalase (CTA1), Phosphoglycerate kinase, Phosphoglyceromutase, Transcription elongation factor, Thiol-specific antioxidant protein, glycerol 3-phosphate dehydrogenase, high-affinity glucose transporter 1, and alcohol dehydrogenase (EhADH2).

As used herein, a “multi-parameter phenotype” refers to a phenotype of a microbial population, as assessed in an individual sample, defined by multiple phenotypic parameters determined by the method described herein. According to the invention, single cells are segmented or distributed into bins based on the intensities of detected phenotypic parameters. The distribution of single cells in bins thus represents a multi-parameter phenotype, also termed “signature”, “fingerprint” or “pattern”, of said microbiota.

As used herein, a “reference profile” is considered a multi-parameter phenotype, signature, pattern or other characterizing representation of a multi-parameter phenotype, that is considered to correlate with or represents an example of any given condition or state. In embodiments, the reference profile is employed in comparison to a measured sample, to assess whether the measured sample at hand exhibits one or more characteristics of the reference profile, and/or whether the measured sample at hand may be characterized as having the state or condition of said reference profile. In embodiments, a reference profile may exhibit multiple bins, discriminatory bins and/or other differentiating features, said bins potentially with thresholds for sufficient numbers or proportions of cells required to be segmented into said bins, in order to characterize any given sample as having the state or condition of said reference profile.

As used herein, “binning” is the process of assigning measured data to discrete, potentially adjoining, categories or units, generally referred to as bins. The term “bin” thus refers to a unit comprising information about the intensities of labels binding to phenotypic parameters of a single cell of the microbiota. Measured data points (preferably for individual cells) are thus assigned to any given bin due to similarities in the measured intensities of labels binding to phenotypic parameters. By way of example, during the analysis a bin will comprise the data points for each bacteria identified to have similar characteristics, thus fulfilling the defining characteristics of the bin, for example each bin will comprise a set of intensities or values for various phenotypic parameters obtained for each cell of the microbiota.

The term “discriminatory bin” shall mean a bin with discriminatory features, which can be used to distinguish states, preferably similar states, for example, two diseases, such as Crohn's disease and Ulcerative colitis. In embodiments, discriminatory features comprise one or more intensities of a label binding a phenotypic parameter of single cell of microbiota, which is representative for a certain state. In embodiments, the segmenting of single cells into bins based on the intensities of detected phenotypic parameters leads to the generation of a multi-parameter phenotype. By way of example, when a sufficient number or proportion of cells are present in any one or more discriminatory bins, the sample may be considered to have the state or condition indicated by said discriminatory bin(s).

In embodiments, a sample comprising microbiota can be any sample comprising microbiota, such as intestinal microbiota, such as a stool sample, saliva, nasopharyngeal swab, skin swab.

In other embodiments, a sample comprising microbiota can be a wastewater sample, food sample, or any liquid or swab sample comprising microbiota obtained from the environment.

In other embodiments, a sample may be any material taken from a subject, a patient, a cell culture of patient cells or cell lines, an animal, or a cell culture of animal cells or cell lines of a biopsy, a blood sample, a tissue sample, or an environmental sample. Basically, any kind of sample that is suspected to contain microbiota. As used herein, the term sample is preferably a biological sample that is obtained or isolated from a subject, i.e. a bodily fluid, tissue or surface (e.g., mucosal swap sample) obtained for the purpose of diagnosis, prognosis, or evaluation of a subject of interest. In case of a liquid sample, or a liquid biopsy the sample may be in embodiments a sample of a bodily fluid, such as blood, serum, plasma, cerebrospinal fluid, urine, saliva, sputum, pleural effusions, a cellular extract, and the like. In further embodiment the sample may be a solid sample, such as a biopsy, a tissue sample, a cell culture sample, cells, a tissue sample, a tissue biopsy, a stool sample or a swap-derived sample.

The term “flow cytometry” or “flow cytometric measurement” refers to an established technique used to detect and measure physical and/or chemical characteristics of a population of cells or particles. Flow cytometric measurement is used for e.g. cell counting, cell sorting, determining cell characteristics and function, detecting microorganisms, detecting biomarker, diagnosing disorders such as blood cancer. In this process, a sample containing cells or particles is suspended in a fluid and injected into a flow cytometer instrument. The sample is focused to ideally flow one cell at a time through a laser beam, where the light scattered is characteristic to the cells and their components. Cells are often labeled with fluorescent markers, so light is absorbed and then emitted in a band of wavelengths. Tens of thousands of cells can be quickly examined, and the data gathered are processed by a computer.

Modern flow cytometers are able to analyze many thousands of particles per second, in “real time” and, if configured as cell sorters, can actively separate and isolate particles with specified optical properties at similar rates.

A flow cytometer typically has five main components: a flow cell, a measuring system, a detector, an amplification system, and a computer for analysis of the signals. The flow cell has a liquid stream (sheath fluid), which carries and aligns the cells so that they pass single file through the light beam for sensing. The measuring system commonly uses measurement of impedance (or conductivity) and optical systems-lamps (mercury, xenon); highpower water-cooled lasers (argon, krypton, dye laser); low-power air-cooled lasers (argon (488 nm), red-HeNe (633 nm), green-HeNe, HeCd (UV)); diode lasers (blue, green, red, violet) resulting in light signals. The detector and analog-to-digital conversion (ADC) system converts analog measurements of forward-scattered light (FSC) and side-scattered light (SSC) as well as dye-specific fluorescence signals into digital signals that can be processed by a computer.

The term “mass cytometry” or “mass cytometric measurement” is a mass spectrometry technique based on inductively coupled plasma mass spectrometry and time of flight mass spectrometry used for the determination of the properties of cells (cytometry). In this approach, antibodies are conjugated with isotopically pure elements, and these antibodies are used to label cellular proteins. Cells are nebulized and sent through an argon plasma, which ionizes the metal-conjugated antibodies. The metal signals are then analyzed by a time-of-flight mass spectrometer. The approach overcomes limitations of spectral overlap in flow cytometry by utilizing discrete isotopes as a reporter system instead of traditional fluorophores which have broad emission spectra.

In embodiments, either flow cytometry or mass cytometry may be employed in the method, using appropriate labels and/or hardware.

The term “segmenting” refers to the characteristic pooling of cells with similar properties into appropriate bins, or bin plots. In embodiments, the pooling of cells into bins is based on the intensities of detected phenotypic parameters, wherein the distribution of single cells in bins represents a multi-parameter phenotype of the microbiota.

In embodiments, the segmenting is performed automatically based on a predetermined algorithm relating to the intensities of detected phenotypic parameters. In embodiments, the segmenting is performed manually based on the intensities of detected phenotypic parameters.

The term “pattern” shall mean a distinctive multi-parameter phenotype as a form for identifying a certain state. The pattern reflects a multi-parameter phenotype of a microbial population. A pattern can be identified that correlates to a certain state. Different patterns can be used to distinguish between different states. A pattern preferably comprises at least one phenotypic parameter associated to a certain state. In embodiments, a pattern comprises a combination of two to eleven phenotypic parameters associated with a certain state. In embodiments, the pattern comprises more than eleven phenotypic parameters associated with a certain state.

The terms “pattern” and “signature” or “fingerprint” may be used interchangeably.

The term “predetermined model”, interchangeable with predictive model, relates to a model comprising a set of associations and/or correlations between distinctive multi-parameter phenotypes with one or more states. In embodiments, a predetermined model can be generated by using a discriminatory feature either from the multi-parameter of phenotypes of the microbiota determined by flow cytometry and/or 16s gene sequencing. In other embodiments, the predetermined model is employed to determine the presence or absence of any given state, by comparing an observed multi-parameter phenotype for any given sample, with the established correlations between multi-parameter phenotypes with one or more states.

The term “state” refers to a defined status or a condition present in a microbial population. In embodiments, the state refers to a medical condition, or condition of wastewater, or any condition of the environment involving microbiota. In embodiments, a medical state is a medical condition, e.g. chronic inflammatory disease. In embodiments, a medical state is preferably an inflammatory bowel disease.

Without limitation, in embodiments, chronic inflammatory disease is selected from the group consisting of inflammatory bowel disease, preferably Crohn's disease and/or ulcerative colitis, rheumatoid arthritis, juvenile arthritis, an IgG4-related disease and/or a kidney disease, such as kidney disease. Preferably, a pattern comprising one or more phenotypic parameters is used for identifying a certain state or distinguishing similar states.

In embodiments, the phenotypic parameter of microbiota labelled in step ii) is a structural feature of a microorganism and/or human immunoglobulin bound to the microorganism.

The term “feature” refers to a detectable structural characteristic that can be identified or measured using the approach described herein. In embodiments, a feature of a microorganism is thus a phenotypic parameter of a microorganism. A feature set refers to a combination of different features. In embodiments, a feature set is a combination of signal intensities of two or more phenotypic parameters, as may be detected by suitable labels.

The term “structural feature of a microorganism” refers to a structural characteristic of a microorganism that can be identified or measured using the approach described herein. For example, a structural feature of a microorganism may be any molecule present on the surface of a microorganism, ranging from a complex structure such as flagella, that propel the organism in aqueous environments, to less sophisticated polysaccharides and proteins.

As used herein, the term “label” in the context of the invention refers to a detection reagent specifically binding to a target, such as phenotypic parameter of microbiota.

The label can be detected using e.g. flow cytometry or mass cytometry, and thus e.g. enable the determination of at least one phenotypic parameter of a microbiota. In one embodiment, the label is a detection reagent which comprises a fluorescent dye and binds specifically to phenotypic parameter of microbiota. In embodiments, the label can be two or more detection reagents binding sequentially to a phenotypic parameter of a microbiota, wherein the detection reagents comprise at least a first detection reagent specifically binding to a phenotypic parameter of microbiota and at least a second detection reagent with a fluorescent dye attaching to the first detection reagent.

The term “labeling” shall mean delivering a binding agent to a cell, for example on a cell surface or into the cell of a microorganism. A labeled sample may refer to a sample in which one or more phenotypic parameters of the cells are labeled with a suitable label, or binding agent.

The present method comprises labeling microbiota with multiple labels, each of which binds a phenotypic parameter of said microbiota. Such labels are subsequently detected in order to quantify said phenotypic parameters. A label preferably comprises a binding agent to provide specific labelling.

In embodiments, a “binding agent” that specifically recognizes a host immunoglobulin or any other structural feature may be selected from, without limitation, an antibody, protein, peptide, nucleic acid, or other small molecule that specifically binds a surface marker or any fragment(s) thereof to identify, track or capture its target molecule.

A label capable of detection in a flow cytometer may be elected by a skilled person without undue effort. In embodiments, the label is a fluorescent label. In embodiments, the fluorescent label is selected from the group consisting of cyanines, Janelia Fluors or rhodamines. Non-limiting examples of cyanines are Cy3 (Cyanine-3), Cy5 (Cyanine-5) and Cy7 (Cyanine-7). Non-limiting examples of rhodamines are TMR, SIR, TMR12 and SiR-d12. Non-limiting examples of Janelia Fluors are Janelia Fluor549-NHS Ester, Janelia Fluor646-NHS Ester, Janelia Fluor585-NHS Ester, Janelia Fluor635-NHS-Ester and Janelia Fluor669-NHS Ester. Further examples of fluorescent labels include, without being limited to, rhodamine and derivatives, lissamine, fluorescein, 5-bromomethylfluorescein and derivatives, DAPI, Hoechst 33258, R-phycocyanin, B-phycoerythrin, R-phycoerythrin, Lucifer Yellow, IAEDANS, 7-Me2N-coumarin-4-acetate, 7-OH-4-CH3-coumarin-3-acetate, monobromobiman, Pyrene trisulfonates such as Cascade Blue and monobromotrimethyl ammoniobiman, Texas Red, Rhodamine Green, Oregon Green 30 488, Oregon Green 514, 7-NH2-4CH3-25-coumarin-3-acetate (AMCA), FAM, TET, CAL Fluor Gold 540, JOE, VIC, Quasar 570, CAL Fluor Orange 560, NED, Oyster 556, TMR, CAL Fluor Red 590, HEX, ROX, LC Red 610, CAL Fluor Red 610, LC Red 610, CAL Fluor Red 610, LC Red 640, CAL Fluor Red 635, LC Red 670, Quasar 670, Oyster 645, LC Red 705, BODIPY FL, Cal Gold, BODIPY R6Gj, Yakima Yellow, Cal Orange, BODIPY TMR-X, JOE, HEX, Quasar-570, TAMRA, Rhodamine Red-X, Redmond Red, BODIPY 581/591, Cy3.5, Cy5, Cy5.5, Cal Red/Texas Red, BODIPY 630/665-X, BODIPY TR-X, Quasar-670/Cy5, Pulsar-650, Dy590, Dy490,Dy 636, Dy682, Atto-488, Atto-532, Atto-Rho-6G, Atto-Rho101, Atto-647N, Atto-680, BMN-488, BMN-505, BMN-536, BMN-562.

The term “immunoglobulin” refers to its ordinary meaning in the art. Immunoglobulins, also known as antibodies, are glycoprotein molecules produced by various immune cells that specifically recognize and bind to particular antigens. The various antibodies are typically classified by isotype, each of which differs in function and antigen responses primarily due to structure variability. Five major antibody classes have been identified in placental mammals: IgA, IgD, IgE, IgG, and IgM. This classification is based on differences in amino acid sequence in the constant region (Fc) of the antibody heavy chains. IgG and IgA are further grouped into subclasses (e.g., in human IgG1, IgG2, IgG3, IgG4, IgA1, and IgA2) based on additional differences in the amino acid heavy chain sequences. Means for detecting immunoglobulins, or detecting specific sub-types of immunoglobulins, are known in the art and can be applied without undue effort.

In embodiments, step ii) comprises additionally using a label that binds a nucleic acid molecule in single cells of the microbiota and selecting cells with a positive nucleic acid molecule signal prior to grouping in step iv). A label binding a nucleic acid molecule refers to any nucleic acid binding reagent, such as a dye or stain, including but not limited to propidium iodide (PI), 7-amino-actinomycin-D (7AAD), acridine orange (AO), hoechst dyes, chromomycin-A2, 4′-6′- diaminido-2-phenylindole (DAPI), cyanine dyes and bromodeoxyuridine (BrdU).

“Chronic inflammatory disease” refers to a medical condition with extended or extended periods of inflammation, typically caused by an unwanted immune response after any endangering infectious or injurious event. For example, in rheumatoid arthritis inflammatory cells and substances attack joint tissue leading to an inflammation that comes and goes and can cause severe damage to joints with pain and potentially deformities. Chronic inflammatory disease includes, without limitation, inflammatory bowel disease, Alzheimer's disease, asthma, cancer, heart disease, rheumatoid arthritis, juvenile arthritis, an IgG4-related disease, kidney disease, ankylosing spondylitis and type 2 diabetes.

“Inflammatory bowel disease” (IBD) comprises any disease comprising inflammation of the gastrointestinal (GI) tract. IBD typically comprises two conditions, Crohn's disease and ulcerative colitis, that are characterized by chronic inflammation of the gastrointestinal (GI) tract.

Crohn's disease (CD) is a disease of chronic inflammation that can involve any part of the gastrointestinal tract. Commonly, the distal portion of the small intestine, i.e., the ileum, and the cecum are affected. In other cases, the disease is confined to the small intestine, colon, or anorectal region. CD occasionally involves the duodenum and stomach, and more rarely the esophagus and oral cavity. The variable clinical manifestations of CD are, in part, a result of the varying anatomic localization of the disease. The most frequent symptoms of CD are abdominal pain, diarrhea, and recurrent fever. CD is commonly associated with intestinal obstruction or fistula, an abnormal passage between diseased loops of bowel. Crohn's disease, like many other chronic inflammatory diseases, can cause a variety of systemic symptoms such as fevers and weight loss. Furthermore, Crohn's disease can affect many other organ systems and cause inflammation of the interior portion of the eye, known as uveitis, or inflammation of the white part of the eye (sclera), a condition called episcleritis. Crohn's disease may result in an increased risk for gallstones, which is due to a decrease in bile acid resorption in the ileum and the bile gets excreted in the stool, or in a type of rheumatologic disease known as seronegative spondyloarthropathy, which is characterized by inflammation of one or more joints (arthritis) or muscle insertions (enteritis). Crohn's disease may also involve the skin, blood, and endocrine system resulting in manifestations such as erythema nodosum, pyoderma gangrenosum, increased risk of blood clots, deep venous thrombosis, autoimmune hemolytic anemia. Additionally, Crohn's disease may cause anemia with the associated symptoms of fatigue, a pale appearance, and other symptoms common in anemia. Crohn's disease can also cause neurological complications, the most common of these are seizures, stroke, myopathy, peripheral neuropathy, headache and depression.

Ulcerative colitis (UC) is a disease of the large intestine characterized by chronic diarrhea with cramping, abdominal pain, rectal bleeding, and loose discharges of blood, pus, and mucus. The manifestations of UC vary widely. A pattern of exacerbations and remissions typifies the clinical course for about 70% of UC patients, although continuous symptoms without remission are present in some patients with UC. Local and systemic complications of UC include arthritis, eye inflammation such as uveitis, skin ulcers, and liver disease. In addition, UC, and especially the long-standing, extensive form of the disease are associated with an increased risk of colon carcinoma.

Colitis is an inflammation of the colon. Colitis may be acute and self-limited or long-term. It broadly fits into the category of digestive diseases. Symptoms of colitis may include: mild to severe abdominal pain and tenderness (depending on the stage of the disease), recurring bloody diarrhea with/without pus in the stools, fecal incontinence, flatulence, fatigue, loss of appetite and unexplained weight loss. In a medical context, the label colitis (without qualification) is used if: The cause of the inflammation in the colon is undetermined; for example, colitis may be applied to Crohn's disease at a time when the diagnosis is unknown, or the context is clear; for example, an individual with ulcerative colitis is talking about their disease with a physician who knows the diagnosis.

Spondyloarthropathy (SpA), or spondyloarthrosis, refers to a joint disease of the vertebral column. As such, it is a class of diseases rather than a single, specific entity. Spondyloarthropathy with inflammation is typically referred to axial spondyloarthritis. The term spondyloarthropathy typically includes joint involvement of the vertebral column from any type of joint disease, including rheumatoid arthritis and osteoarthritis, but the term is also used for a specific group of disorders with certain features, which are often termed seronegative spondylarthropathies, which show an increased incidence of HLA-B27, as well as negative rheumatoid factor and antinuclear antibodies (ANAs, also known as antinuclear factor or ANF), which are autoantibodies that bind to the cell nucleus or contents of the cell nucleus.

Rheumatoid arthritis (RA), is a well-known long-term autoimmune disorder that primarily affects joints, typically resulting in inflamed and painful joints. Most commonly, the wrist and hands are involved, and the disease may also affect other parts of the body, including skin, eyes, lungs, heart, nerves, and blood. The underlying mechanism involves the body's immune system attacking the joints, resulting in inflammation.

IgG4-related diseases (IgG4-RD), also known as IgG4-related systemic disease, is a chronic inflammatory condition characterized by tissue infiltration with lymphocytes and IgG4-secreting plasma cells, and various degrees of fibrosis. In the majority of people with this disease, serum IgG4 concentrations are elevated during an acute phase. Diagnosis is typically made due to the presence of painless swellings or due to complications such as jaundice, due to involvement of the pancreas, biliary tree or liver.

Juvenile idiopathic arthritis (JIA), also known as juvenile rheumatoid arthritis (JRA), is a common chronic rheumatic disease of childhood, typically with onset before 16 years of age. JIA is an autoimmune, noninfective, inflammatory joint disease, characterized by chronic joint inflammation. JIA is a subset of childhood arthritis, but unlike other, more transient forms of childhood arthritis, JIA persists, typically for at least six weeks, and in some subjects is a lifelong condition.

Systemic lupus erythematosus (SLE), also known as Lupus, is an autoimmune disease in which the body's immune system attacks healthy tissue in many parts of the body. Symptoms vary and may be mild to severe, including painful and swollen joints, fever, chest pain, hair loss, mouth ulcers, swollen lymph nodes and feeling tired. The mechanism involves an immune response by autoantibodies against a person's own tissues, commonly involving anti-nuclear antibodies and inflammation. Several variants of lupus are known, including, without limitation, erythematosus including discoid lupus erythematosus, neonatal lupus, and subacute cutaneous lupus erythematosus.

The terms “individual,” “subject,” or “patient” typically refer to humans, but also to other animals including, e.g., other primates, rodents, canines, felines, equines, ovines, porcines, mice and the like.

The term “a medical condition associated with microbiota” refers to any medical condition caused by changes in microbiota or causing a change or imbalance in the microbiota of a subject. A medical condition associated with microbiota is typically, although not necessarily, associated with inflammation. In embodiments, a medical condition associated with microbiota is chronic inflammatory disease, inflammatory bowel disease, rheumatoid arthritis, juvenile arthritis, an IgG4-related disease and/or a kidney disease, such as chronic kidney disease.

In embodiments, the method described herein comprises (a) training a machine-learning model on one or more correlations between any given multi-parameter phenotype and a particular state, or between two or more states in a subject, and (b) segmenting of single cells into bins based on the intensities of detected phenotypic parameters.

The term “machine learning” refers to a field of inquiry devoted to understanding and building methods that ‘learn’, that is, methods that leverage data to improve performance on some set of tasks. It is seen as a part of artificial intelligence. Machine learning algorithms build a model based on sample data, known as training data, in order to make predictions or decisions without being explicitly programmed to do so.

A “machine-learning model” has, for example, been trained to recognize certain types of patterns. A “machine-learning model” is typically trained with a set of data and thus enables the development and recognition of correlations between particular data points and states or conditions. In embodiments of the invention, the set of data for training shall comprise the bins, for example discriminatory bins, for characterizing a state, or distinguishing similar states. The trained model is able to recognize any particular pattern, based either on the multi-parameter phenotype as a whole or parts thereof, such as discriminatory bins, and predict an associated state.

In embodiments, a “random forest” approach or machine learning model or algorithm is applied. A random forest approach is typically considered a classification algorithm consisting of many decisions trees. By way of example, it typically uses bagging and feature randomness when building each individual tree to try to create an uncorrelated forest of trees whose prediction by committee is more accurate than that of any individual tree. Random forest is a supervised learning algorithm. The “forest” it builds is an ensemble of decision trees, usually trained with the “bagging” method. Put simply, random forest builds multiple decision trees and merges them together to get a more accurate and stable prediction. The general idea of the bagging method is that a combination of learning models increases the overall result. Random forest is a flexible, easy-to-use machine learning algorithm with high reliability. It is also one of the most-used algorithms, due to its simplicity and diversity, and can be used for both classification and regression tasks. Selecting an appropriate model is within the ability of a skilled person.

Particular aspects of the present invention may be computer-implemented. Accordingly in embodiments the present method may be a computer-implemented method. The person skilled in the art is aware of which aspects and features of the present invention may be computer-implemented. In a further aspect the present invention relates to a computer-readable storage device, comprising a software or computer program product suitable to carry out the invention. In a further aspect the present invention relates to a computer-readable storage medium having stored thereon the software or computer program product according to the invention, or suitable to conduct the method or other aspects of the invention.

As disclosed herein, the method can be used to diagnose or prognose a disease, or to determine the risk of a subject to developing any given disease. As used herein, “diagnosis” in the context of the present invention relates to the recognition and detection of a clinical condition. In embodiments, the assessment of severity may be encompassed by the term “diagnosis”. “Prognosis” relates to the prediction of an outcome or a specific risk for a subject. This may also include an estimation of the chance of recovery or the chance of an adverse outcome for said subject.

The methods of the invention may also be used for monitoring, therapy monitoring, therapy guidance and/or therapy control. “Monitoring” relates to keeping track of a patient and potentially occurring complications, e.g. to analyze the progression of the healing process or the influence of a particular treatment or therapy on the health state of the patient. The term “therapy monitoring” or “therapy control” in the context of the present invention refers to the monitoring and/or adjustment of a therapeutic treatment of said patient, for example by obtaining feedback on the efficacy of the therapy. As used herein, the term “therapy guidance” refers to application of certain therapies, therapeutic actions or medical interventions based on the value/level of one or more biomarkers and/or clinical parameter and/or clinical scores. This includes the adjustment of a therapy or the discontinuation of a therapy. In the present invention, the term “risk assessment” relates to the grouping of subjects into different risk groups according to their further prognosis. Risk assessment also relates to stratification for applying preventive and/or therapeutic measures.

As used herein, the terms “comprising” and “including” or grammatical variants thereof are to be taken as specifying the stated features, integers, steps, or components but do not preclude the addition of one or more additional features, integer, steps, components or groups thereof. This term encompasses the terms “consisting of” and “consisting essentially of”. Thus, the term “comprising”, “including” or “having” mean that any further component (or likewise features, integers, steps and the like) can or may be present. The term “consisting of” means that no further component (or likewise feature, integers, steps and the like) is present.

### FIGURES

The following figures are presented to describe particular embodiments of the invention, without being limiting in scope.

### BRIEF DESCRIPTION OF THE FIGURES

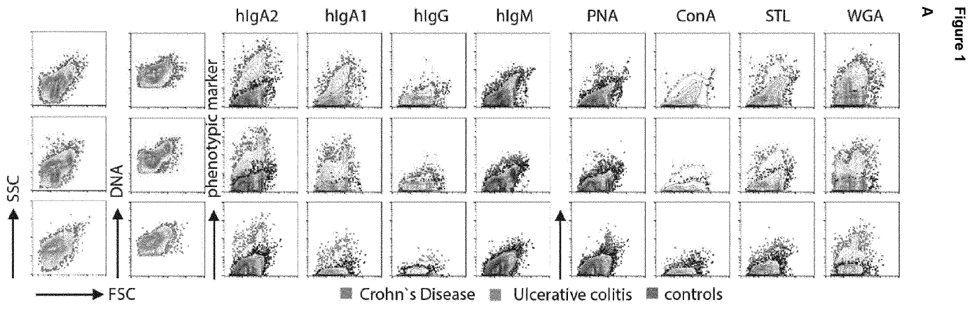

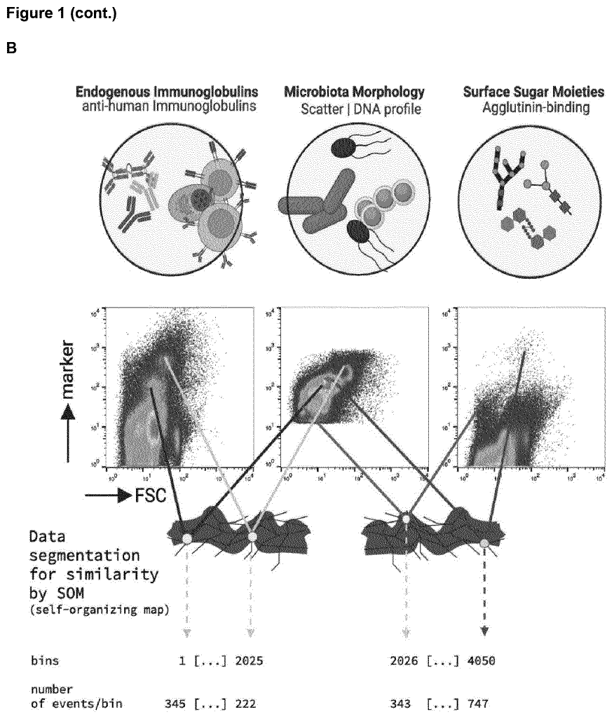

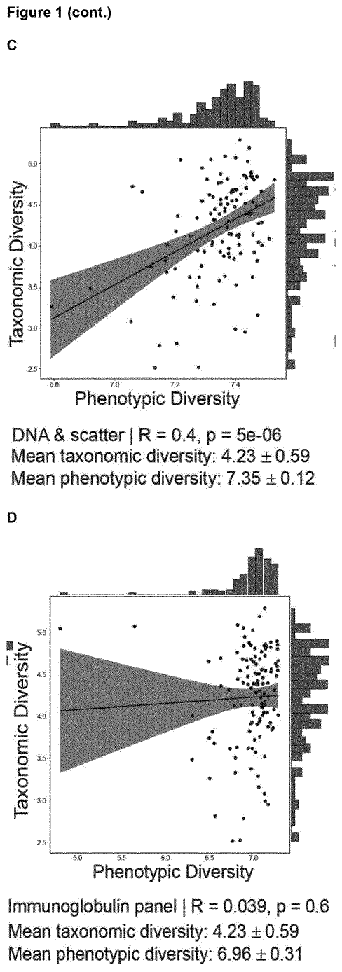

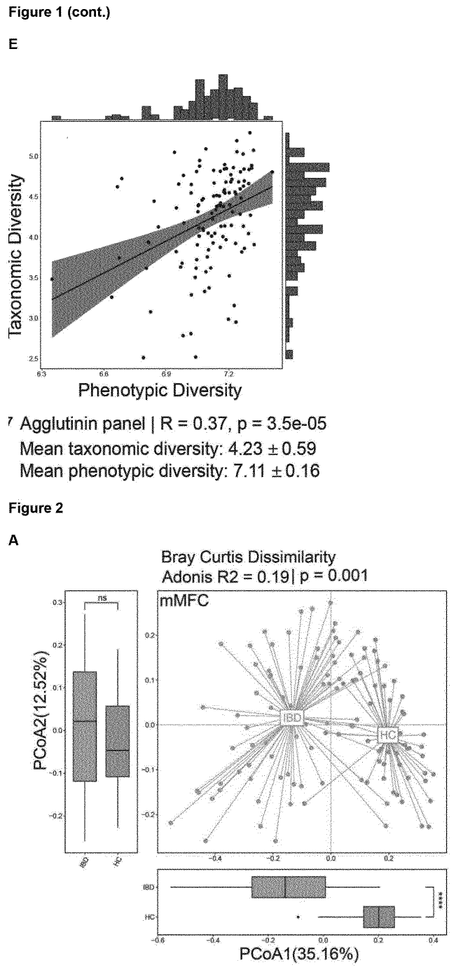

FIG. 1: Multi-parameter microbiota flow cytometry.

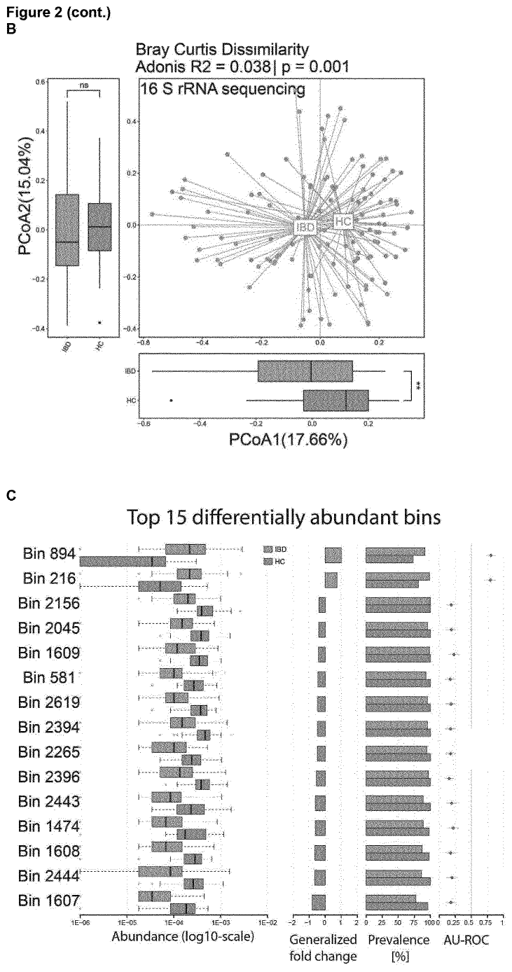

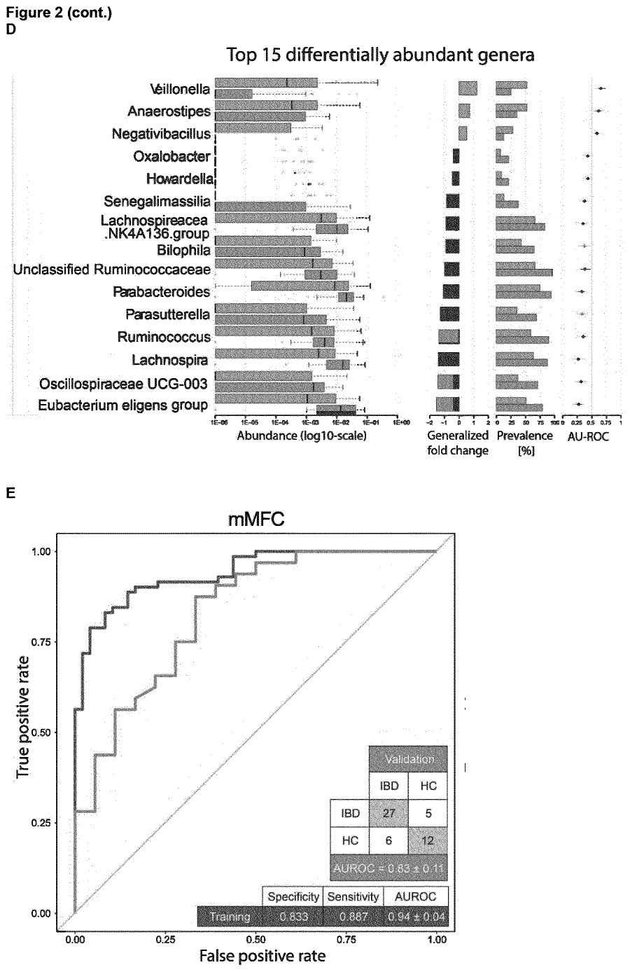

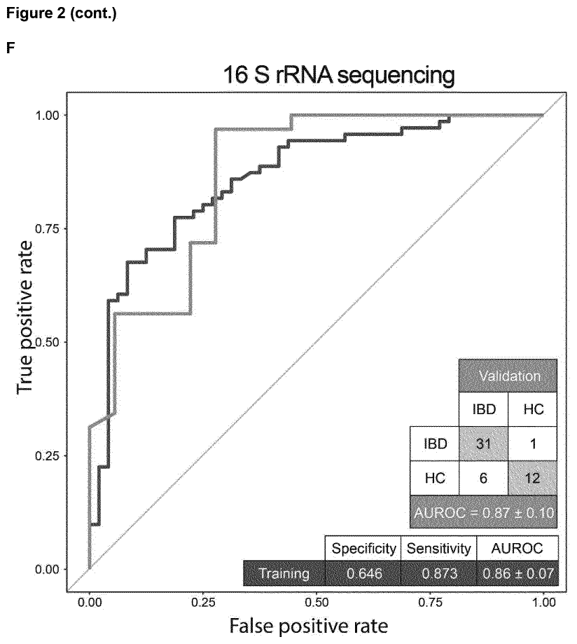

FIG. 2: Phenotypic classification of IBD patients' microbiota by random forest models.

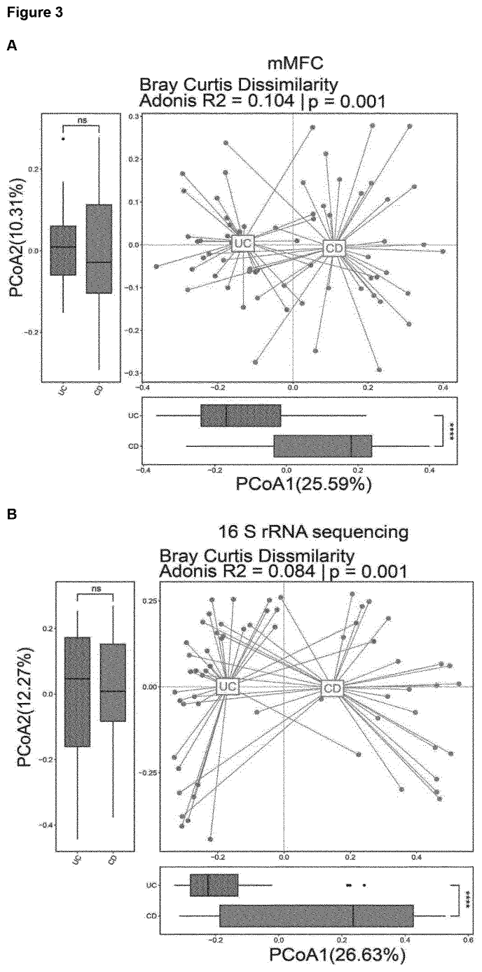

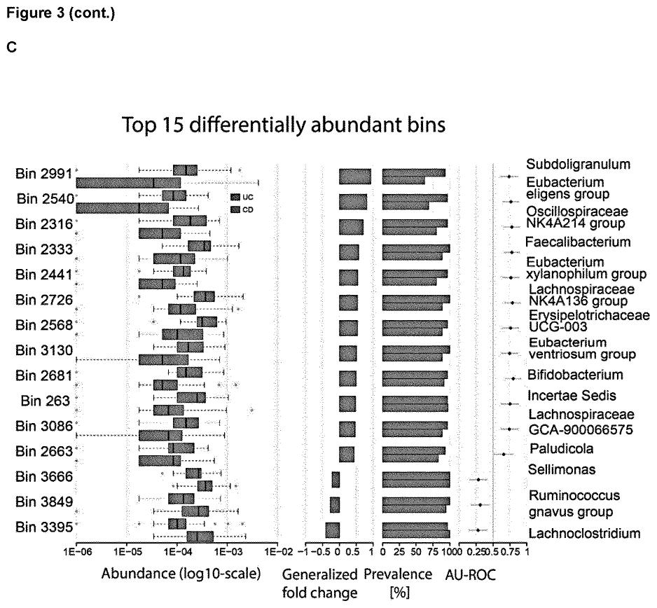

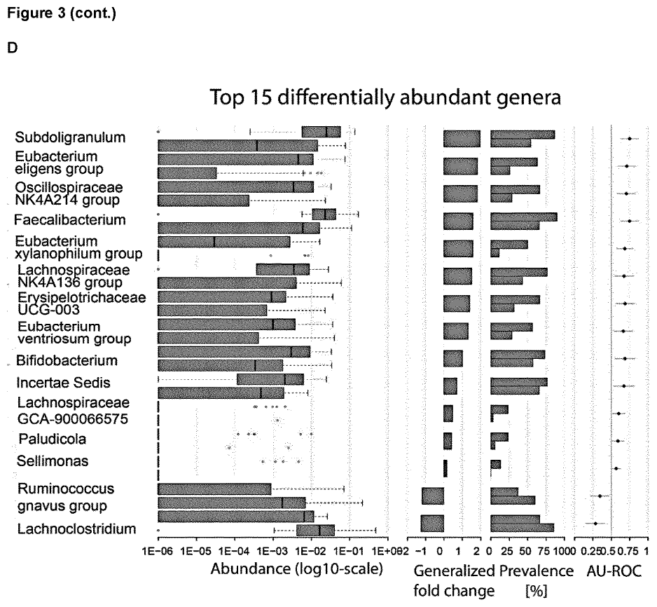

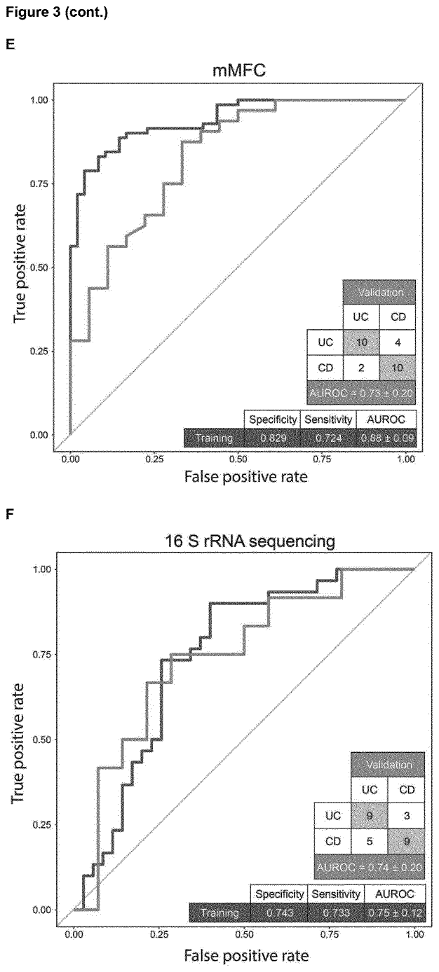

FIG. 3: IBD sub-classification via specific microbiota phenotypes in CD (Crohn's Disease) and UC (Ulcerative Colitis).

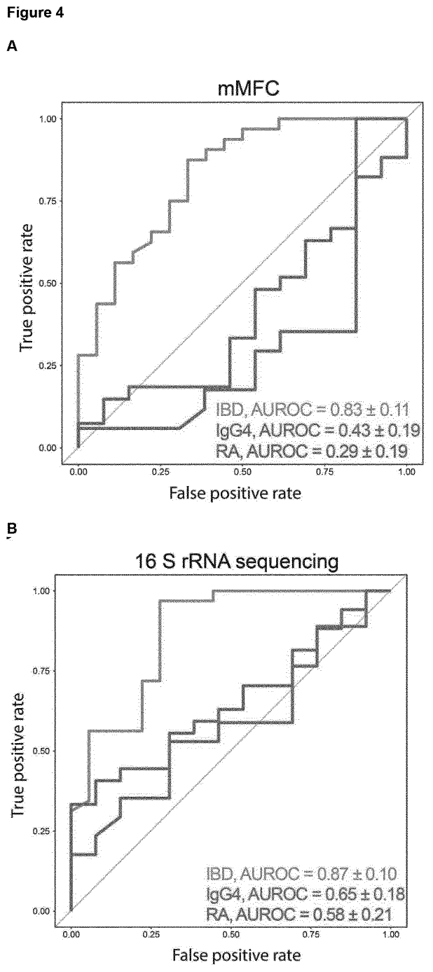

FIG. 4: Phenotypic sample classification is specific.

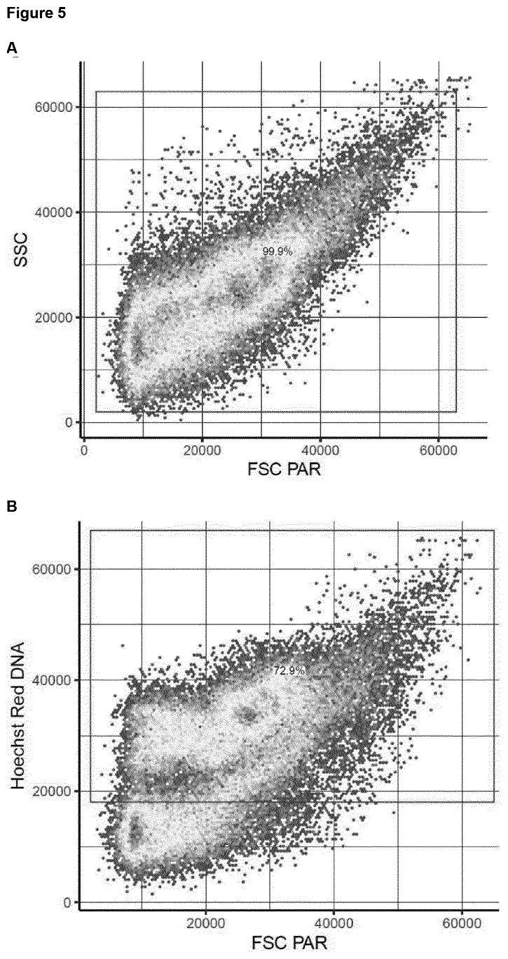

FIG. 5: Automated gating prior to SOM segmentation.

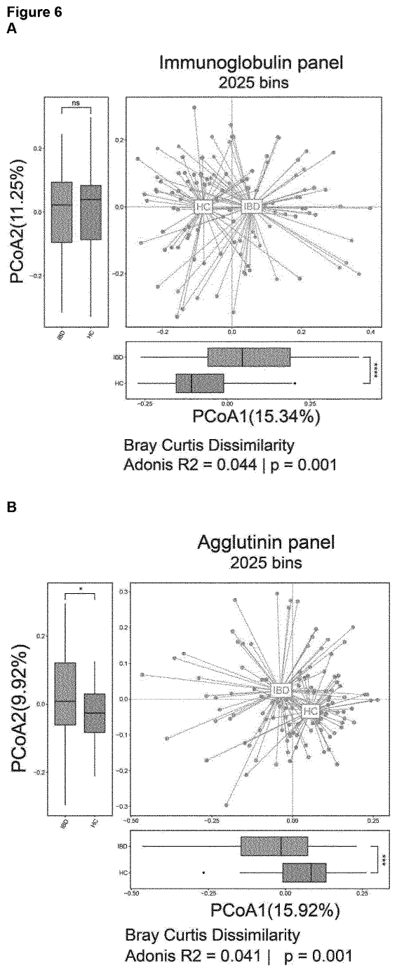

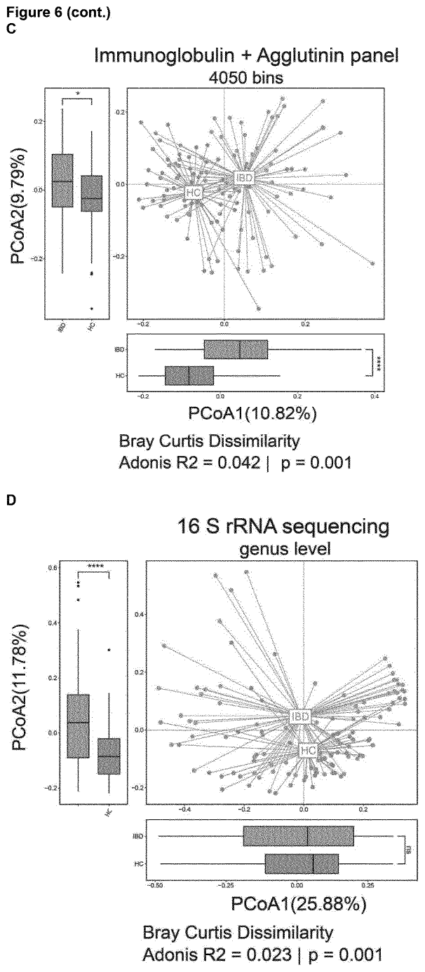

FIG. 6: Phenotypic and taxonomic alterations in IBD.

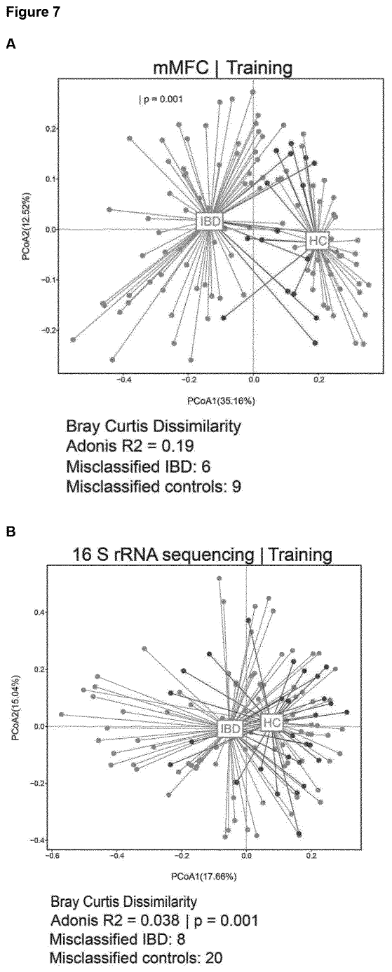

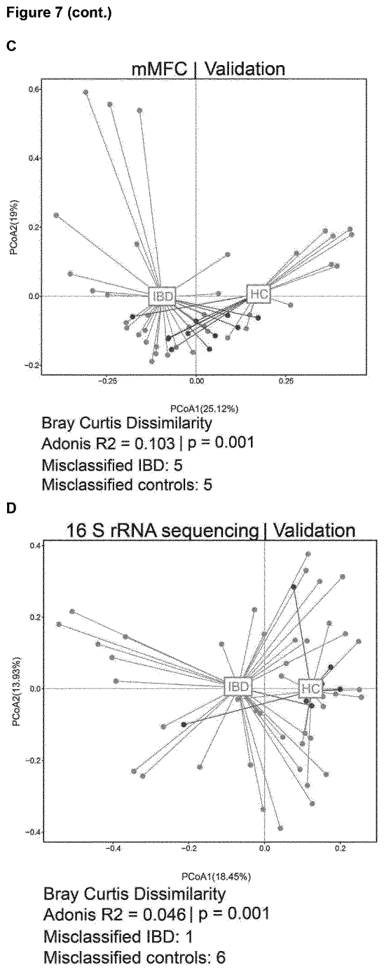

FIG. 7: Marker combination of discriminatory bins in mMFC for classification.

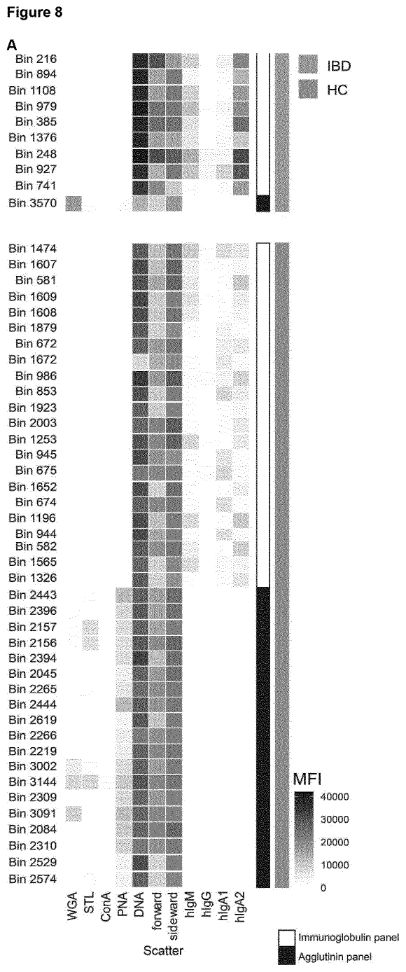

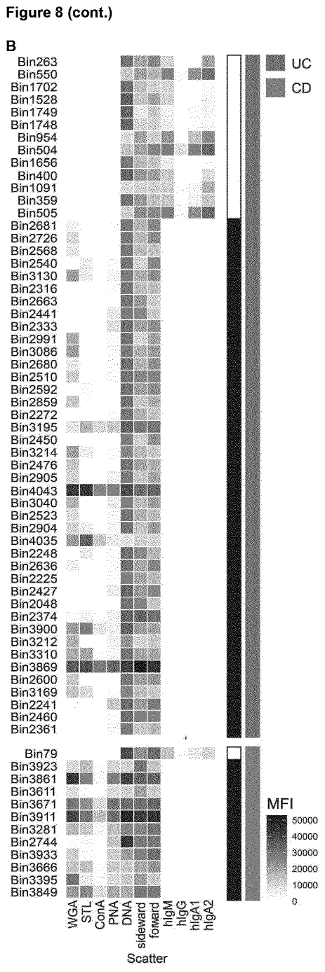

FIG. 8: Location of misclassified samples for the classification of IBD samples and controls projected on PCoA plot for dissimilarity (Bray-Curtis) of their microbiota phenotype (mMFC) or the microbiome composition (16 S rRNA sequencing).

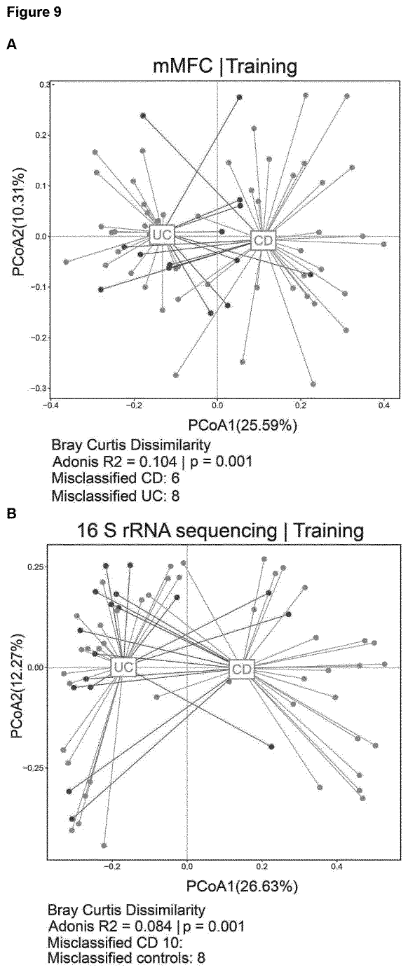

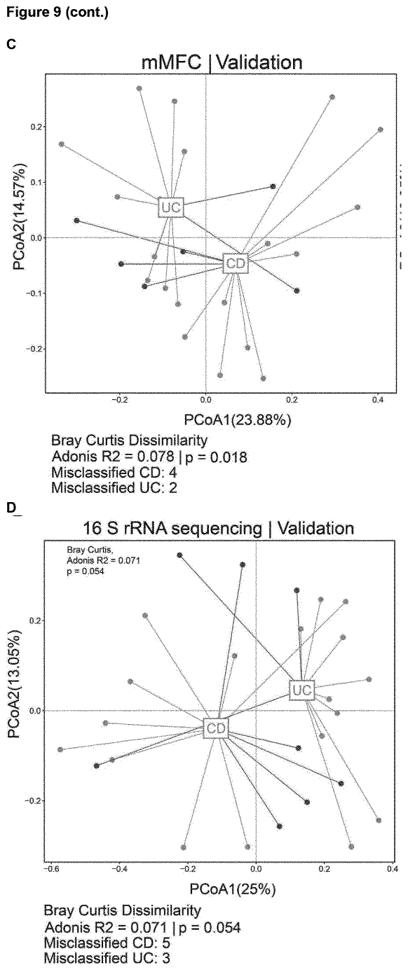

FIG. 9: Location of misclassified samples for the classification of CD and UC samples projected on PCoA plot for dissimilarity (Bray-Curtis) of their microbiota phenotype (mMFC) or the microbiome composition (16 S rRNA sequencing).

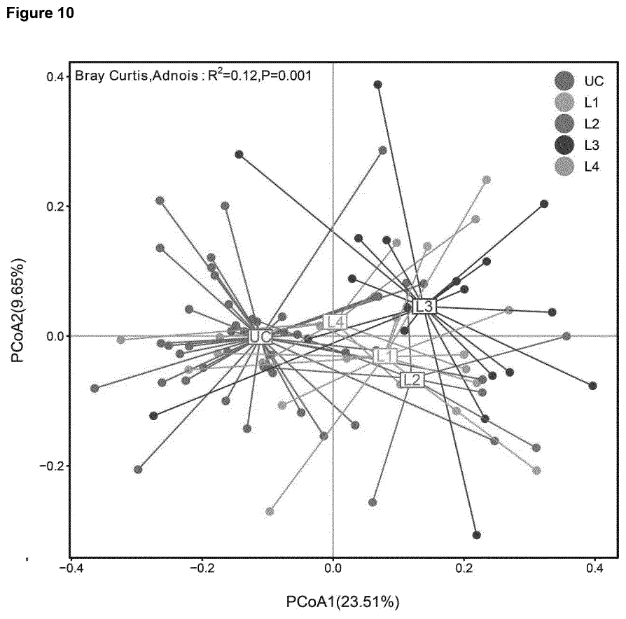

FIG. 10: Impact of inflammation site on Crohn's disease on microbiota phenotype diversity metrics.

FIG. 11: Microbiota phenotypic signatures in inflammatory diseases and healthy controls.

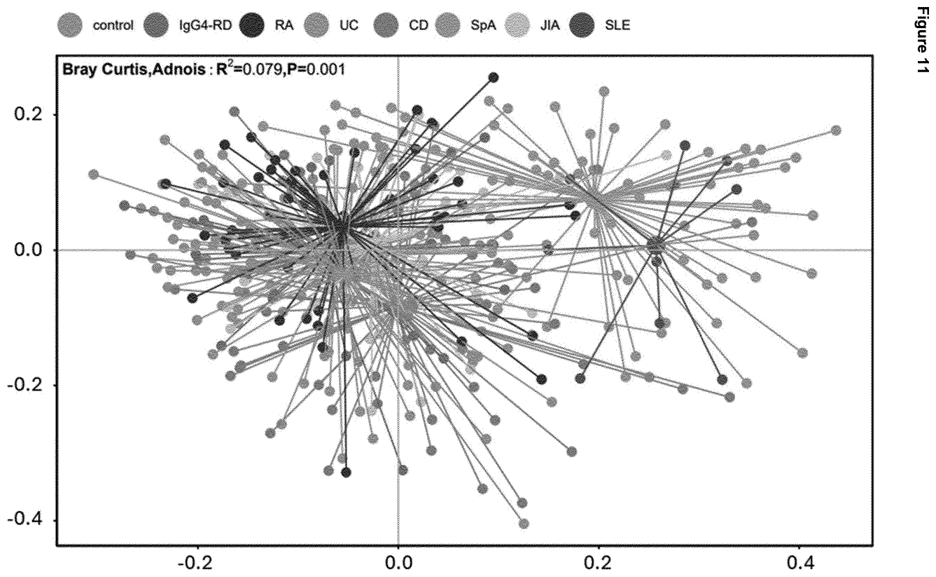

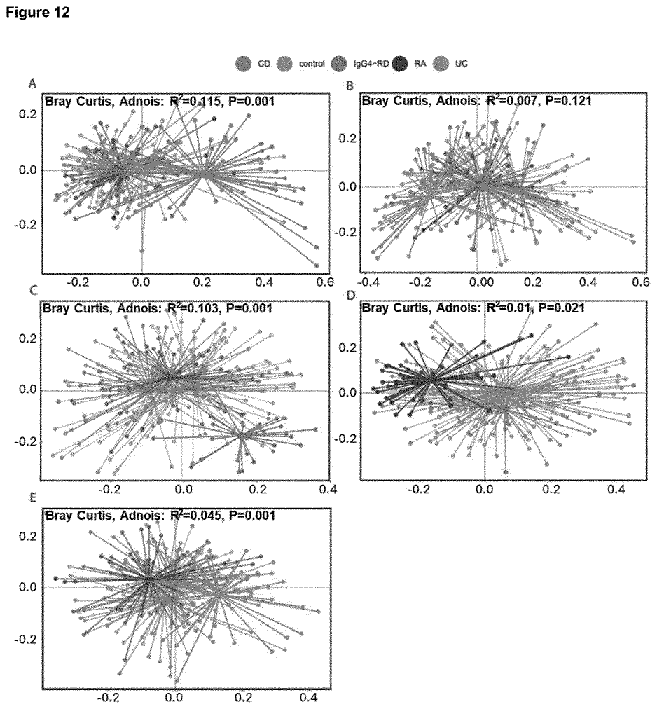

FIG. 12: Selective phenotypic signatures distinguish disease entities.

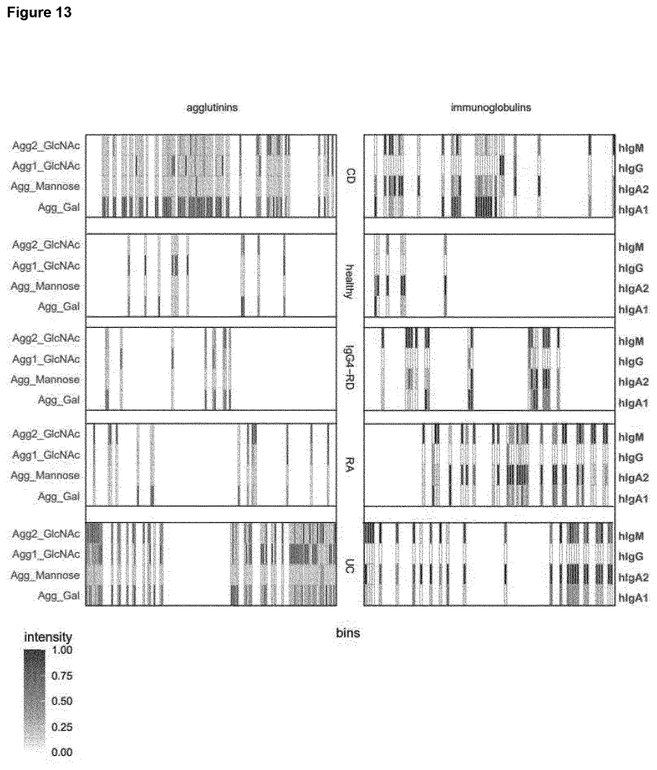

FIG. 13: Disease-specific variation in microbiota phenotypic signatures.

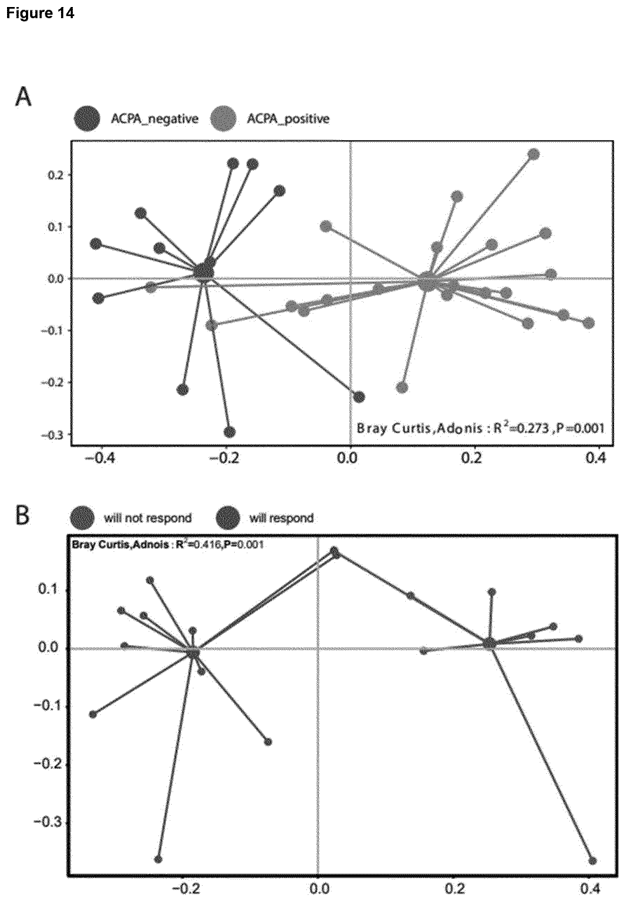

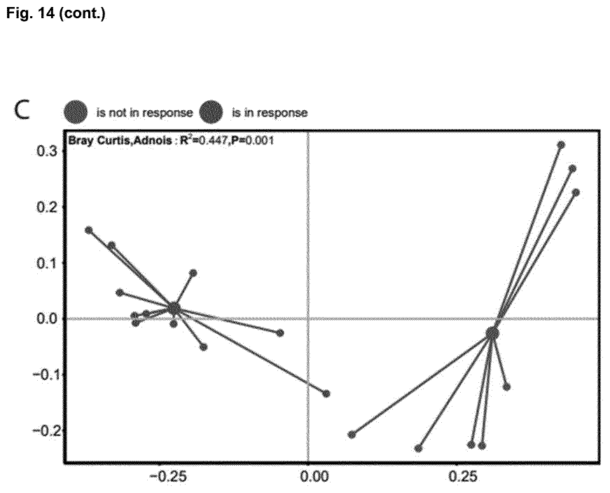

FIG. 14: Microbiota phenotypic signature enables stratification and monitoring in patients with distinct diseases.

### DETAILED DESCRIPTION OF THE FIGURES

FIG. 1: Multi-parameter microbiota flow cytometry. A Exemplary contour plots illustrating the staining patterns of each acquired parameter when stained in combination for either the immunoglobulin panel (hIgA1/2, hIgG, hIgM) or the agglutinin panel (WGA: Wheat Germ Agglutinin, PNA: Peanut Agglutinin, STL: Solanum Tuberosum Agglutinin, ConA: Concanavalin A) for UC (purple), CD (green) and healthy control's (grey) microbiota samples. In black the individual background signal of the only DNA dye sample is indicated. B Schematic overview of data processing from the flow cytometric data files (.fcs) of one sample to a data table that comprises 7 phenotypic features per bin and the abundance of events in that range of parameters. For comparison of microbiota phenotypes, the inventors compute Bray-Curtis dissimilarity and project the result onto PCoA here exemplary shown for a smaller cohort of controls, Crohn's disease and ulcerative colitis patients. C, D, E Correlation of phenotypic diversity obtained by microbiota flow cytometry with 2025 clusters computed on the DNA profile (C, R=0.4, p=0.000005), the immunoglobulin panel (D, R=0.039, p=0.67) and the lectin panel (E, R=0.37, p=0.000035), respectively, to the taxonomic diversity observed by 16 S rRNA sequencing (Pearson correlation).

FIG. 2: Phenotypic classification of IBD patients' microbiota by random forest models. Cohort composition for the entire approach: 103 IBD samples (CD: 49, UC: 54), 66 sex- and age-matched controls. IBD: yellow, Controls: grey. The cohort was initially split randomly at a 70/30 ratio. 70% of the data were used as training set to compute the SOM, select features for classification and train the model. The residual 30% served as validation set which the SOM was only mapped to and the criteria for modelling just transferred, not re-computed. A PCoA of the dissimilarity (Bray-Curtis) all samples for selected, significant features for cohort discrimination with 51 mMFC bins and B 37 genera from 16 S rRNA sequencing. Statistics: Wilcoxon. C List of top 15 discriminatory bins from RFE feature selection for cohort classification by mMFC evaluated on the entire training set for abundance, generalized fold change (determines order), prevalence and individual prediction score. D List of top 15 discriminatory genera identified by RFE feature selection for cohort identification (IBD vs. controls) evaluated on the entire training set for abundance, generalized fold change (determines order), prevalence and individual prediction score. E Performance of the prediction model based on microbiota phenotype (mMFC) in training stage (blue): 70% of data (random sampling, 71 IBD samples, 48 control samples) with 10 times repeated 10-fold cross-validation evaluated by AUROC and model performance parameters (specificity, sensitivity). Model performance in validation stage (green): residual 30% of data (32 IBD samples, 18 control samples) evaluated by AUROC and the respective confusion matrix. F Performance of the prediction model in training stage (blue): 70% of data (similar to mMFC model) with 10 times repeated 10-fold cross-validation evaluated by AUROC and model performance parameters (specificity, sensitivity). Model performance in validation stage (green): residual 30% of data (similar to mMFC model) evaluated by AUROC and the respective confusion matrix.

FIG. 3: IBD sub-classification via specific microbiota phenotypes in CD (Crohn's Disease) and UC (Ulcerative Colitis). Cohort composition based on the previous classification of samples as IBD for the microbiota phenotype obtained by mMFC (90, CD: 49, UC: 41) and 16 S rRNA sequencing (91, CD: 49, UC: 42). The samples applied in both methods are not matching at that stage. CD: purple, UC: green. A PCoA projection of samples classified as IBD based on intestinal microbiota phenotype for dissimilarity (Bray-Curtis) in 65 mMFC bins selected for significant discrimination of CD and UC. Statistics: Wilcoxon. B PCoA projection of dissimilarity (Bray-Curtis) of samples classified as IBD based on microbiome composition regarding 28 genera significantly different between CD and UC samples. Statistic: Wilcoxon. C Corresponding list of top 15 discriminatory bins from the RFE feature selection evaluated on the training set for abundance, generalized fold change (determines the order), prevalence in UC and CD microbiota and individual prediction score (AUC) for classification. D Corresponding list of top 15 discriminatory genera from the RFE feature selection evaluated on the training set for abundance, generalized fold change (determines the order), prevalence in UC and CD microbiomes and individual prediction score (AUC) for classification. E Performance of the prediction model based on flow cytometric data: training stage (blue): 70% of data (random sampling) with 10 times repeated 10-fold cross-validation and validation stage (green): residual 30% of data with respective confusion matrix. AU-ROC, sensitivity and specificity are indicated at default threshold (0.5). F Performance of the prediction model based on taxonomic data: training stage (blue): 70% of data (random sampling, different then mMFC data) with 10 times repeated 10-fold cross-validation and validation stage (green): residual 30% of data with respective confusion matrix. AU-ROC, sensitivity and specificity are indicated at default threshold (0.5).

FIG. 4: Phenotypic sample classification is specific. Each model (mMFC-based, 16 S rRNA-based) trained to classify IBD and controls was used to classify a set of samples from patients of rheumatoid arthritis (RA, 17 samples) and IgG4-related diseases (IgG4-RD, 27 samples), plus 13 controls. A Prediction performance of IBD-trained microbiota phenotype model. B Prediction performance of a 16 S rRNA-based IBD-model (genus level).

FIG. 5: Automated gating prior to SOM segmentation. A, B Exemplary FACS plots illustrating the gating strategy in the R package FlowCore before data segmentation by SOM. The microbiota phenotype regardless of additional markers applied was always determined on DNA positive events (B).

FIG. 6: Phenotypic and taxonomic alterations in IBD. Stool samples of 103 IBD patients and 66 controls were simultaneously characterized by mMFC and 16 s rRNA sequencing to describe IBD-related alterations in the intestinal microbiota and compare the capacity of both methods to capture cohort-specific microbiota/microbiome features prior to any further data processing e. g. feature selection. For mMFC the contribution of each panel is outlined as well as their combination is outlined. IBD: yellow, Controls: grey. A PCoA projection (Bray-Curtis dissimilarity) of the flow cytometric phenotype in regards of endogenous immunoglobulins detected on the bacterial surface represented with 2025 bins. Statistics: Wilcoxon. B PCoA projection (Bray-Curtis dissimilarity) of samples dissimilarity for 2025 clusters on the agglutinin panel-based phenotype. Statistics: Wilcoxon. C PCoA projection (Bray-Curtis dissimilarity) of each sample's microbiota phenotype for the combination of the immunoglobulin and agglutinin panel (4050 clusters). Statistics: Wilcoxon. D PCoA projection (Bray-Curtis dissimilarity) of the taxonomic dissimilarity on genus level. Statistics: Wilcoxon.

FIG. 7: Marker combination of discriminatory bins in mMFC for classification. List of bins required for the classifications shown in FIG. 2 (IBD: yellow, controls: grey) and FIG. 3 (CD patients: purple, UC patients: green) and the respective composition of marker intensities (from the flow cytometry data) per bin (human immunoglobulins-white, Agglutinins-black). Both panels rely on DNA staining of the microbiota and scatter information. The assignment of bins to a cohort was determined by computing the fold change of the number per events per bin between the classified cohorts. A Marker intensities per bin for the 51 discriminatory bins to classify IBD patients (yellow) from controls (grey). Bin with the highest discrimination potential is highlighted (red box). B Marker intensities per bin for the 65 discriminatory bins to classify CD patients (green) from UC patients (purple). Bins with the highest discriminatory potential are highlighted (red box).

FIG. 8: Location of misclassified samples for the classification of IBD samples and controls projected on PCoA plot for dissimilarity (Bray-Curtis) of their microbiota phenotype (mMFC) or the microbiome composition (16 S rRNA sequencing). Highlighting the locations of misclassified samples in either training or the validation stage of modelling for both methods applied to characterize the samples (mMFC, 16 S rRNA sequencing). Misclassified samples are highlighted in purple and connected to their respective cohort. Number of misclassified samples are given in the plot. A Misclassified samples of the model classifying IBD and controls by microbiota phenotype in training stage (total samples IBD: 71, misclassified IBD samples: 6, total controls: 48, misclassified control samples: 9). B Misclassified samples of the model classifying IBD and controls by microbiome composition in training stage (total IBD samples: 71, misclassified IBD samples: 11, total controls: 48, misclassified control samples: 17). C Misclassified samples of the model classifying IBD and controls by microbiota phenotype in validation stage (total IBD samples: 32, misclassified IBD samples: 5, total controls: 18, misclassified control samples: 6). D Misclassified samples of the model classifying IBD and controls by microbiome composition in validation stage (total IBD samples: 32, misclassified IBD samples: 1, total controls: 18, misclassified control samples: 6).

FIG. 9: Location of misclassified samples for the classification of CD and UC samples projected on PCoA plot for dissimilarity (Bray-Curtis) of their microbiota phenotype (mMFC) or the microbiome composition (16 S rRNA sequencing). Highlighting the locations of misclassified samples in either training or the validation stage of modelling for both methods applied to characterize the samples (mMFC, 16 S rRNA sequencing). Misclassified samples are highlighted in purple and connected to their respective cohort. Number of misclassified samples are given in the plot. At this stage of modelling the samples between mMFC and 16 S rRNA sequencing did not necessarily match. A Misclassified samples of the model classifying CD and UC samples by microbiota phenotype in training stage (total samples: 64, CD: 35, misclassified CD: 6, UC: 29, misclassified UC: 8). B Misclassified samples of the model classifying IBD and controls by microbiome composition in training stage (total samples: 65, CD: 35, misclassified CD: 10, UC: 30, misclassified UC: 8). C Misclassified samples of the model classifying IBD and controls by microbiota phenotype in validation stage (total samples: 26, CD: 14, misclassified CD: 4, UC: 12 misclassified UC: 2). D Misclassified samples of the model classifying IBD and controls by microbiome composition in validation stage (total samples: 26, CD: 14, misclassified CD: 5, UC: 12 misclassified UC: 3).

FIG. 10: Impact of inflammation site on Crohn's disease on microbiota phenotype diversity metrics. All IBD patients are shown. PCoA for CD and UC (red) samples after feature selection with indication of the inflammation site in CD. L1: terminal ileum (yellow), L2: colonic (green), L3: ileocolonic (dark blue), L4: gastrointestinal (light blue).

FIG. 11: Microbiota phenotypic signatures in inflammatory diseases and healthy controls. Principal Coordinate plot illustrating the microbiota phenotypic signatures of individuals with Crohn's disease (CD), Ulcerative colitis (UC), Spondyloarthropathy (SpA), rheumatoid arthritis (RA), IgG4-related diseases (IgG4-RD), juvenile idiopathic arthritis (JIA), systemic lupus erythematosus (SLE), and healthy controls. Each dot represents an individual donor, color-coded by disease cohort, and connected to the respective centroid, indicating their phenotypic difference based on Bray-Curtis distance.

FIG. 12: Selective phenotypic signatures distinguish disease entities. Identification of disease-specific phenotypic signatures by selecting bins differentiating between two classes, i.e., one disease versus the rest. The resultant selective phenotypic signatures defining specific disease entities or states are individually plotted against others for (A) Crohn's Disease (CD) patients, (B) Healthy Controls, (C) IgG4-Related Diseases (IgG4-RD), (D) Rheumatoid Arthritis (RA), and (E) Ulcerative Colitis (UC) patients.

FIG. 13: Disease-specific variation in microbiota phenotypic signatures. The phenotypic signature, characterized by varying combinations and intensities of surface staining in different bins, exhibits distinctive patterns for each disease entity or state.

FIG. 14: Microbiota phenotypic signature enables stratification and monitoring in patients with distinct diseases. The phenotypic microbiota signature effectively stratifies patients within a disease entity or state. (A) Rheumatoid arthritis (RA) patients can be classified based on the presence of autoantibodies against citrullinated proteins (ACPA). Notably, 16S rRNA gene sequencing failed to differentiate ACPA-positive and ACPA-negative RA patients (data not shown). (B, C) Multi-parameter microbiota flow cytometry can be applied to (B) predict and (C) monitor the response of CD patients to anti-TNF therapy. In (B), samples were taken before the start of the therapy and retrospectively analyzed. In (C), samples were taken and analyzed after 6 weeks of anti-TNF therapy.

### EXAMPLES

The following examples are presented to describe particular and potentially preferred embodiments of the invention, without being limiting in scope, thereby providing support for the workability of the invention.

The human intestinal microbiota is associated with disease, yet to date the analysis of the composition has not resulted in major diagnostic or therapeutic improvements. The Examples presented below introduce multi-parameter microbiota flow cytometry (mMFC) combined with a machine learning pipeline as a tool for rapid, single cell analysis of intestinal microbiota revealing phenotypic, disease-specific microbiota signatures.

With mMFC, the inventors characterized phenotypic parameters of the intestinal microbiota, in particular coating with host-immunoglobulins and expression of distinct surface sugars moieties, from stool samples of 103 IBD patients and 66 age- and sex-matched healthy controls. The multi-parametric signature of individual samples for sample-wise comparison and disease classification was benchmarked against 16S rRNA gene-based microbiome profiling.

The phenotypic microbiota signature reliably distinguished patients with inflammatory bowel diseases (IBD) from healthy controls (AUROC=0.94±0.04 versus AUROC=0.86±0.07 based on 16S rRNA gene sequencing) and further, was distinct from that of rheumatoid arthritis (RA) and IgG4-related disease (IgG4-RD) when 16S rRNA sequencing did not reveal such clear differences. Within the IBD cohort, mMFC profiling robustly classified Crohn's disease (CD) and ulcerative colitis (UC) patients (AUROC=0.88±0.09 versus AUROC=0.75±0.12 by 16S rRNA gene sequencing).

Overall, the inventors demonstrate single-cell bacterial phenotyping by mMFC, advancing disease classification with high potential for applications in clinical settings and offering a novel, direct approach to evaluate microbiota properties relevant in host-microbiota interaction in disease.

### Example 1

### Microbiota Phenotyping Integrates Taxonomy-independent Microbiota Properties

The inventors collected stool samples of 103 individuals with IBD (CD: n=49, UC: n=54), diverse in disease activity and treatment regimens, and 66 age- and sex-matched healthy controls. All samples were subjected to mMFC and 16S rRNA gene sequencing. For mMFC, bacterial cells from stool samples were stained with two individual staining panels targeting host immunoglobulin coating with the antibody isotypes IgA1, IgA2, IgG and IgM and different surface sugar moieties recognized by peanut agglutinin (PNA), Concanavalin A (ConA), Solanum tuberosum agglutinin (STA) and wheat germ agglutinin (WGA), respectively (FIG. 1 A). In addition, quantitative DNA staining with Hoechst 33342 and light scatter properties [16, 19] were used to define the single cell-based microbial phenotypic signatures of an individual (FIG. 1 A).

For analysis of the mMFC data, the inventors applied a self-organizing map (SOM) [24]. First, bacterial cells were distinguished from non-cellular debris and instrument noise by gating for DNA-positive events (FIG. 5A, B). From the pre-gated data and for each staining panel, the inventors sub-sampled 10.000 events of each sample and concatenated them into one dataset representing all bacterial phenotypes found in this cohort. The inventors then applied the SOM algorithm to the combined dataset, segmenting the dataset into 2025 bins, each comprising phenotypically similar cells, for each staining panel. The resulting bin segmentation matrix was subsequently applied back to all samples individually, each containing 3×10⁵ events (FIG. 1B).

The abundance of cells in each bin was used to compute dissimilarity and diversity metrics for the direct comparison between the flow cytometric signature of individual samples to their corresponding 16S rRNA gene sequencing results. The Shannon diversity index determined by mMFC with the SOM pipeline, only taking into account DNA content and scatter properties, correlated with the Shannon diversity index of 16S rRNA gene sequencing in the inventors dataset (Pearson's r=0.4, p=5×10⁻⁶) as previously shown by Props et al. [18]. Likewise, the agglutinin staining signature (Agglutinin panel: Pearson's r=0.37, p=3.5×10⁻⁵) of mMFC and 16S rRNA gene sequencing diversity showed correlation. In contrast, no correlation in Shannon diversity could be seen between the immunoglobulin-coating signature and taxonomic diversity (Pearson's r=0.039, p=0.67) (FIG. 1 C-E). Correspondingly, the immunoglobulin panel increased inter-sample variance in the mMFC data compared to the agglutinin panel or the DNA profile (Immunoglobulin panel mean phenotypic diversity: 4.32±0.56; DNA profile mean phenotypic diversity: 7.35±0.12; Agglutinin panel mean phenotypic diversity: 7.11±0.16).

### Example 2

### Microbiota Phenotypes and Microbial Taxonomy of IBD Patients Allow Disease Classification

To compare individual phenotypic microbiote signatures of IBD patients and healthy controls, the inventors projected the Bray-Curtis dissimilarity between all samples using a principal coordinate analysis (PCoA) plot (FIG. 1 B). Using the 2025 bins representing either host immunoglobulin coating or agglutinin staining, samples of IBD patients and healthy donors differed significantly (R²=0.044 and 0.041, respectively) with PCoA1 accounting for 15.34% and 15.92% of the data variation, respectively (FIG. 6 A, B). Agglutinin staining also showed significant dissimilarity in PCoA2, which accounted for an additional 9.9% of the data variation (FIG. 6 B). Combination of the immunoglobulin coating and agglutinin panels to a total of 4050 bins, resulted in an intermediate dissimilarity (R²=0.042) (FIG. 6 C) with both PCoA dimensions capturing significant phenotypic differences between IBD samples and controls. 16S rRNA gene sequencing and Bray-Curtis dissimilarity projection using taxonomic data (genus level) also showed compositional differences in the samples of IBD patients and healthy donors (R²=0.023). A significant difference on taxonomic level between IBD patients and healthy donors was found only in PCoA2, accounting for 13.42% of data variance, but not in PCoA1 accounting for 25.8% of the data (FIG. 6 D).

To test whether the phenotypic microbiota signature can be used to discriminate IBD patients from healthy controls on an individual basis, the inventors randomly divided the samples into a training set comprising 70% of the samples and a validation set consisting of the remaining 30% of the samples. The training set was used for data segmentation by SOM and for the selection of bins for classification and training the machine learning model. Both, SOM and classification model of the training cohort were directly projected onto the validation set without re-computing. The same samples were used for model training and validation using the 16S rRNA gene sequencing profiles for benchmarking.

Prior to model training, discriminatory features were selected as bins with a statistically significant difference between the IBD cohort versus healthy cohort (p<0.05, Wilcoxon). The p-value was corrected for multiple testing (Bonferri-Holm), before feature selection by the Recursive Feature Elimination (RFE [25]) algorithm. 51 phenotypic bins relevant for cohort discrimination with mMFC were identified (FIG. 7 A). PCoA projection using the selected set of bins indicated that the dissimilarity between IBD patients and healthy donors by mMFC increased from to R²=0.19 from R²=0.041 including all 4050 bins (FIG. 2 A and FIG. 6 A), allowing for significant cohort separation in PCoA1 (35.1% data variance). In the 16S rRNA gene sequencing profiles, 37 bacterial genera were identified, best discriminating IBD patients from healthy donors. Restriction to these genera improved the discrimination between IBD patients and healthy donors (before: R²=0.023, after: R²=0.038, FIG. 2 B and FIG. 6 D).

The top 15 discriminatory bins or bacterial genera identified by the feature selection (RFE) are shown in FIG. 2C-D together with their respective abundance in each sample, their prevalence in each cohort, the generalized fold change and AU-ROC value for the prediction of cohort identity, ordered by fold change.

The bins used for the discriminatory mMFC model were represented in most samples with a prevalence ranging from 75-100% (FIG. 2 C for top 15 bins and data not shown). For example, the two discriminatory bins 894 and 216 identifying the IBD phenotype displayed an up to 10 times higher abundance in IBD samples and comprised events coated by host IgM, IgA1 and IgA2 and having high DNA and scatter signals (FIG. 7 A). The other discriminatory MFC bins within the top 15 were more abundant in the healthy cohort comprising bins from both the immunoglobulin-coating and agglutinin panel (FIG. 7 A). The three differentially abundant genera Veillonella, Anaerostipes and Negativibacillus identified by 16 s rRNA sequencing were more abundant in IBD patients. The other discriminatory genera showed a relative decrease in IBD patients (FIG. 2 D).

In contrast to the bins reflecting the mMFC data, some genera discriminating between IBD patients and healthy controls were detectable in only a few samples, e. g. Oxalbacter and Howardella (FIG. 2 D showing top 15 genera). Overall, the prevalence of discriminatory genera ranged from 5-80%.

Using the selected features derived from mMFC or 16S rRNA sequencing, respectively, the inventors generated random forest models for automated sample classification. Using the mMFC-based model, samples of the training set were correctly classified with an AUROC of 0.94±0.04, with a sensitivity of 0.887 and a specificity of 0.833. In validation, samples were classified correctly with an AUROC of 0.83±0.11, misclassifying 11 out of 50 samples (6 HC, 5 IBD). 16S rRNA gene sequencing had classified samples with an AUROC=0.86±0.07 (specificity: 0.646, sensitivity: 0.873) in the training set (7 misclassifications, 6 HC, 1 IBD) and an AUROC=0.87±0.10 in the validation phase (8 misclassifications, 3 IBD, 4 HC). In total, 28 out of the 169 samples were misclassified by the mMFC model (training: 17/119, testing: 11/50) and 35 samples were misclassified by the 16S rRNA gene model (training: 28/109, testing: 6/50) while more healthy controls were misclassified as IBD than vice versa (mMFC: 13 IBD, 15 HC; 16 S rRNA: 12 IBD, 23 HC).

### Example 3

### Distinct Phenotypic and Taxonomic Signatures in CD and UC

Next, the inventors investigated whether mMFC or 16S rRNA gene sequencing allowed the further classification of IBD samples into CD and UC. To this end, the inventors used the samples correctly classified as IBD in the respective models, i.e., n=90, 49 CD and 41 UC for mMFC and n=91, 49 CD and 42 UC for 16S rRNA gene sequencing. Samples were no longer identical between mMFC and 16S rRNA gene sequencing since the results from the first modelling stage had not been identical. Again, each of the respective cohorts was again split into a training set (70% of the samples), used for the selection of discriminatory features between CD and UC and model training, and a sample set (30% of the samples) used for validation.

Discriminatory bin and genera selection was again applied on the full set of 4050 bins and full 16S gene sequencing data, respectively, to identify features discriminating between CD and UC. The inventors identified 65 mMFC bins (PCoA R²=0.104, FIG. 3 A; FIG. 7 B) and 28 genera (PCoA R²=0.084, FIG. 3 B). For both sets of features, CD and UC samples were significantly different in PCoA1 which equally represented approx. 25% of the data variation (mMFC: 25.59%, 16 S rRNA sequencing: 26.63%, FIG. 3 A, B).

For the discrimination between CD and UC by mMFC, bins derived from the agglutinin panel were highly relevant for cohort discrimination (51 out of 65) (FIG. 3 C and FIG. 7 B). 53 out of the 65 bins were more abundant in the UC cohort (FIG. 7 B). The top 15 discriminatory bins of the RFE selection defined by mMFC had a high prevalence in each sample ranging from 60-100% among all samples, while the top 15 discriminating genera were found in only in 2-80% of the samples (FIG. 3 C, D). The mMFC bins distinguishing CD from UC were distinct from those discriminating IBD patients and healthy controls (FIG. 2 C and 3 C, supp. FIG. 3 A, B). In contrast, certain genera, such as Eubacterium eligens group and the Lachnospiraceae NK4A 136 groups were among the top discriminators of CD versus UC, as well as the ones discriminating IBD patients and healthy controls (FIG. 2 D and 3 D). Using the selected features, the inventors trained models with the respective training sample sets for CD and UC classification. The mMFC-based model generated with the training set of 54 samples resulted in an AUROC=0.88±0.09, with 8 CD and 6 UC samples being misclassified (FIG. 9 A). The 16S rRNA gene-based model using the 55 training samples resulted in an AUROC=0.75±0.12 (FIG. 3 F), with 10 CD and 8 UC sample misclassifications. Sensitivity was similar in both models (mMFC: 0.724, 16 S: 0.733), but the mMFC-based model had higher specificity (mMFC: 0.829 versus 16 S: 0.743). Validation of the models gave similar AUROCs for both methods: 0.73±0.2 with mMFC phenotyping and 0.74±0.2 with 16S taxonomic profiling. Within the set of validation, mMFC misclassified 2 CD and 4 UC samples and the 16S rRNA gene sequencing 5 CD and 3 UC samples.

### Example 4

### Phenotypic Microbiota Alterations in IBD Are Not Classifying Other Chronic Inflammatory Diseases

To determine whether the microbiota phenotype discriminating IBD patients and healthy controls merely reflects a state of chronic inflammation and its associated dysbiosis as such, or whether it is characteristic for IBD, the inventors applied the IBD classification model to samples from patients with other chronic inflammatory diseases (CIDs), namely rheumatoid arthritis (RA, n=17) and IgG4-related disease (IgG4-RD, n=27) and a new set of healthy controls (n=13). For this, the inventors mapped the SOM previously generated using the IBD and healthy samples onto the additional samples and applied the model using the bins selected to classify IBD samples as described above (FIG. 2 E, AUROC: 0.83±0.11). The RA and IgG4-RD samples were not classified as IBD (RA cohort AUROC: 0.29±0.19; IgG4-RD AUROC: 0.43±0.19) (FIG. 4 A). The inventors also tested whether the 16S rRNA gene sequencing-based signature was specific for IBD by submitting the same samples to the taxonomic classification model. The 16S rRNA gene sequencing-based taxonomic discriminators of the IBD microbiome (AUC 0.87±0.10) were less distinct, i.e. mainly alike random association (RA AUROC: 0.58±0.21, IgG4-RD AUROC: 0.65±0.18) (FIG. 4 B).

### Example 5

### Distinct Microbiota Signatures in Chronic Inflammatory Diseases and Healthy Controls

As seen in FIG. 11-14 , various chronic inflammatory disease (CID) entities and healthy controls were analyzed using multi-parameter microbiota flow cytometry. Stool samples were collected and processed, with subsequent staining for immunologically relevant protein and sugar structures on bacterial cell surfaces using fluorescence-labeled antibodies and agglutinins. The data reveal that clear distinctions between the microbial populations obtained from patients with IBD, but also Spondyloarthropathy (SpA), rheumatoid arthritis (RA), IgG4-related diseases (IgG4-RD), juvenile idiopathic arthritis (JIA), systemic lupus erythematosus (SLE), and healthy controls, was possible, thus enabling employment of the technology beyond diseases of the digestive tract, for example to other medical conditions with an inflammatory component.

Additionally, quantitative staining for DNA content was performed. Flow cytometry analysis determined the multi-parametric signature of each individual's intestinal microbiota. The resulting multi-dimensional data was clustered into 4050 bins based on phenotypic similarity of single cells, enabling the identification of phenotypic signatures associated with specific disease entities or disease states.

### Discussion of the Examples:

In this study, the inventors introduce the phenotypic characterization of single bacterial cells in complex intestinal communities using an advanced multi-parameter microbiota flow cytometry (mMFC) approach acquiring quantitative DNA staining, light scattering, host-immunoglobulin coating, and expression of defined sugar residues on the surface of the bacteria. The inventors combine mMFC with data segmentation by a SOM, machine learning for feature selection and random forest modelling for automated, unsupervised sample classification.

To demonstrate the feasibility of the single-cell-based microbiota analysis approach, the inventors have applied it to the analysis of stool samples of a cohort of patients with IBD and healthy controls. The mMFC-based microbiota phenotyping is able to reliably classify stool samples of IBD patients and healthy donors, and further allows for the distinction of CD and UC patients within the IBD cohort. In comparison, the respective 16S rRNA gene sequencing-based classification models were less well able to discriminate IBD from healthy controls and performed similarly in the classification of CD and UC patients. Of note and in contrast to the taxonomy-based models, the discriminatory features found with mMFC were different for classifying IBD or UC and CD, highlighting similarities and dissimilarities of both disease subclasses relevant in diagnosis and therapy.

The performance of the 16S-based models in the present cohort of n=103 (49 UC and 54 CD samples) was comparable to other microbiome-based modelling attempts, such as that of Linares-Blanco et al. who have analysed larger cohorts (642 individuals, IBD: 321) [26]. This probably indicates a general limitation of 16S rRNA gene sequencing-based models that may not be overcome by enlarging cohorts [5, 6, 27, 28]. A major limitation of 16S rRNA gene sequencing approaches is the large cross-sectional heterogeneity of microbiomes, which hinders the identification of disease-specific taxonomic groups on the one hand, and on the other, the applicability of taxonomic identifiers to all members of a cohort or all patients suffering from a disease. In addition, taxonomic variation between healthy donors and IBD patients are also influenced by total microbial load [18, 28], which is rarely considered in microbiome studies. In fact, models which base the distinction of healthy versus IBD and even CD versus UC solely on microbial and fungal load perform similarly to taxonomy-based differentiation models [29].

Ultimately, it is of question if disease-specific, common taxonomic alterations exist and should be further targeted for clinical exploitation of the human microbiota.

mMFC captures the taxonomic diversity of a microbial community by quantitative DNA staining and scatter properties (FIG. 1 C) [16, 18, 19], and the bacterial phenotypes, here defined by way of example by host-immunoglobulin coating and surface sugar moiety expression. The mucosal immunoglobulin response in IBD is an important factor in disease pathology and may link the microbiota and their pathogenic role. It has been proposed that IgA is preferentially bound to colitogenic bacteria in IBD [13] and that IgA1 and IgA2 have distinct binding patterns to bacteria in CD and UC [30]. Moreover, the presence of IgG directed against intestinal bacteria has been associated with IBD [14, 31]. Altered surface glycosylation of bacteria could reflect on metabolic activity, nutritional state and an inflammatory microenvironment resulting in impaired interaction between bacteria or with the host [32]. The present concept is supported by data which show increased functional changes rather than taxonomic changes in IBD, which mMFC captures by bacterial phenotyping rather than taxonomy [12, 33]. Recently, it has also been shown that binding of host lectins, such as intelectin-1, to bacteria contributes to the pathogenesis of intestinal inflammation [34]. Thus, mMFC extends the (taxonomic) community composition by additional functional parameters and host reaction in microbiota analysis as this data indicate that the pattern of antibody coating and surface sugar expression can represent disease-specific phenotypes which reflect host responses and alterations in the gut microenvironment. In line with this, the phenotypic signature of IBD patients was clearly distinct from patients suffering rheumatoid arthritis and IgG4-related diseases. This was in contrast to 16S rRNA gene sequencing, which showed a taxonomic signature in IBD patients rather similar to that of rheumatoid arthritis and IgG4-related diseases, indicating that most taxonomic alterations reflect chronic-inflammatory conditions in general and are not specific to a particular inflammatory disease [35]. Also, the identified disease-specific phenotypes in this study did not correlate with the 16S rRNA gene sequencing profiles, as a high similarity in abundance of populations with specific phenotypes within donor groups was found, which have a low similarity in their taxonomic microbiome composition.

Within the IBD cohort, the phenotypic microbiota signature could be used to classify UC and CD samples regardless of disease state (clinically active vs. remission) and type of therapy. Of the original 4050 phenotypic clusters, the inventors identified 51 bins required to identify IBD patients and 65 bins required to classify UC and CD patients. Repeatedly, there was no overlap in the bins classifying IBD and UC/CD, pointing to traceable differences in the UC- and CD-specific microbiota phenotypes and to potentially different roles of the microbiota in both diseases. With mMFC these different phenotypes can now be isolated directly by fluorescent-activated cell sorting and investigated functionally. Although it is known that different regions of the intestine also differ in microbial composition [36], the inventors did not observe that the distinction between CD and UC was driven by the site of inflammation. The present model was not trained to distinguish sites of inflammation and as such did not stratify patients according to the Montreal classification L1-L4 (FIG. 10). Accordingly, the inventors did not observe increased misclassification of CD patients with colonic (L2) involvement, with the limitation that the sample number of L2 CD patients was too small to show this formally.

Of particular note is that the multi-parameter analysis of microbial populations also allowed the identification and characterization of disease states not directly involved in the digestive tract or intestinal health. As shown above, the characterization of disease states is not limited to IBD but enables identification of unique multi-parameter microbial signatures for various inflammatory diseases. The data reveal that clear distinctions between the microbial populations obtained from patients with IBD, but also Spondyloarthropathy (SpA), rheumatoid arthritis (RA), IgG4-related diseases (IgG4-RD), juvenile idiopathic arthritis (JIA), systemic lupus erythematosus (SLE), and healthy controls, was possible, thus enabling employment of the technology to identify and characterize not only diseases of the digestive tract, but also other medical conditions, for example those with an inflammatory component, and further to non-medical bacterial populations, as disclosed by way of example above.

Although final conclusions about the causal relationship between microbiota phenotype and disease pathogenesis cannot be drawn per se, the present flow cytometric approach as such includes the option of isolating microbiota populations of interest directly from the complex sample for detailed taxonomic analysis or functional studies [37]. The inventors mMFC protocol works for life bacteria and set-ups for the direct isolation via cell sorting of strictly anaerobic bacteria has already been described [38].

Altogether, the inventors demonstrate that single-cell phenotyping of bacteria by multi-parametric microbiota flow cytometry (mMFC) is a potent tool for the investigation and characterization of complex microbial communities, such as the intestinal microbiota for the identification of disease-specific bacterial signatures and generates robust results compared to 16S rRNA sequencing.

Additionally considering the reduced cost and processing time for data acquisition and analysis, mMFC presents a non-invasive, rapid, cost-effective alternative and supplement to “conventional” microbiota profiling with the potential to be applied for point-of-care diagnostics and therapy monitoring, but also to investigate the role of defined bacteria in disease pathogenesis.

### Methods:

### Stool Samples

Stool samples were provided by all donors under approval of the local ethics committee of the Charité Berlin (approval reference: EA4/014/20; EA2/113/20) and in accordance with the Helsinki II Declaration. The samples were taken with stool sampling tubes (Sterilin®, VWR Cat. No. 215-0327) and transferred to 4° C. instantly upon arrival.

### Patient and Public Involvement

During patient recruitment, the study was openly and transparently communicated by informed patient consent form. Processed study data was made available at any stage of the study to participants. Healthy controls completed a questionnaire assessing potential confounders such as age, gender, smoking status, and nutritional habits. The control group did not report any chronic intestinal inflammation. The patients' disease state and entity were indicated by the examining clinicians. The sample collection logistics was designed to enable easy participation in the study e. g. from home. Members of the research team are actively involved in science communication events to explain the methods and goals of this study to the broad public and to receive feedback regarding patients' needs and concerns.

### Stool Sample Processing

Stool samples were kept at 4° C. for a maximum of 96 h before processing, if longer storage was required samples were frozen directly at −20 or −80° C. The sample was diluted in autoclaved and 0.2 μm sterile-filtered PBS (in-house, Steritop® Millipore Express®PLUS 0.22 μm, Cat. No: 2GPT05RE) according to weight in the ratio of 100 mg/ml and homogenized by vortexing. The suspension was subsequently filtered through 70 μm (Falcon, Cat. No. 352350) and 30 μm filters (CellTrics®, Sysmex, Cat. No. 04-0042-2316) and the faecal supernatant got separated from the cell pellet at 13,000×g (15 min, 4° C.). The cell pellet was re-suspended in the initial sample volume (100 mg/ml). 10 μl were stored directly at −20° C. for 16 S rRNA gene sequencing. Cell density (OD) obtained at 680 nm at a VIS-spectrometer (Multiskan™ FC, Thermo Scientific™) and for each sample a set of equal stocks at 0.4 OD/ml and 0.8 OD/ml were prepared for long-term storage by re-suspending the respective volume from the microbiota suspension in 1 ml 40 % glycerol/LB freezing medium and immediate transfer to −80° C.

Staining Microbiota for Multi-parameter Microbiota Flow Cytometry (mmfc)

Frozen microbiota stocks (0.4-0.8 OD/ml) were topped up with 1 mL of autoclaved and sterile-filtered PBS and the freezing medium was removed after centrifugation at 13,000×g for 10 min, 4° C. The pellet was incubated in 500 μl PBS containing isotype controls of later applied detection antibodies (mIgG1: 20 μg/ml, mIgG 2a: 10 μg/ml) for 15 min, 4° C. The blocking step was topped with 1.5 ml PBS and subjected to another centrifugation step (13,000×g, 10 min, 4° C.). The supernatant was discarded, and pellets re-suspended in a DNase containing PBS buffer (PBS/0.2 % BSA/25 μg/μl DNase, Sigma Aldrich Cat. No. 10104159001), which was used for the entire staining protocol as follows. Cell density was adjusted to obtain tests with 0.02-0.04 OD/ml. Staining for human immunoglobulins was performed in 100 μL per test with 1:100 (v/v) of each detection antibody: anti-human IgM-Brilliant Violet 650 (clone: MHM-88, Biolegend® Cat. No. 314526), anti-human IgG-PE/Dazzle™ 594 (clone: HP6017, Biolegend® Cat. No. 409324), anti-human IgA1-Alexa Fluor 647 (clone: B3506B4, Southern Biotech Cat. No. 9130-31), anti-human IgA2-Alexa Fluor 488 (clone: A9604D2, Southern Biotech Cat. No. 9140-30). Staining for bacterial sugar moieties was performed in 100 ul per test with 0.5 μg/test Peanut Agglutinin-CFR 488 (PNA, Biotium Cat. No. 29060), 0.5 μg/test Concanavalin A-CFR®680 (Con A, Biotium Cat. No. 29020-1) and 0.25 μg/test Wheat Germ Agglutinin-CFR®555 (WGA, Biotium Cat. No. 29076-1); 0.5 μg/test of biotinylated Solanum Tuberosum Agglutinin (STL, Vector Laboratories/Biozol Cat. No. B-1165) were shortly pre-incubated with 2 μL (1:50, v/v) anti-Biotin-PerCP antibody (clone: Bio3-18E7, Miltenyi Cat. No. 130-133-293) before adding to the residual reagents. The tests were incubated for 30 min at 4° C. and subsequently topped up with 1 ml 5 μM Hoechst solution (Hoechst 33342, Thermo Fisher Scientific Cat. No. 62249) for another 30 min at 4° C. After incubation the tests were washed with 900 μl PBS/BSA at 13,000×g and re-suspended in fresh PBS/BSA for acquisition.

### Microbiota Flow Cytometry

BD Influx® cell sorter was applied for all cytometric measurements. The sheath buffer (PBS) for the instrument was autoclaved and sterile filtered (Steritop® Millipore Express®PLUS 0.22 μm, Cat. No: 2GPT05RE) before each fluidics start up. The quality and reproducibility of each acquisition was assured by the alignment of lasers, laser delays and laser intensities by Sphero™ Rainbow Particles (BD Biosciences Cat. No. 559123) and control of scatter properties by Megamix-Plus FSC beads (BioCytex Cat. No7802). Samples were acquired with an event rate below 15,000 events. In each acquisition 300,000 DNA positive events were recorded. 16S rRNA sequencing (Illumina MiSeq platform)

For 16 S rRNA sequencing the inventors amplified the V3/V4 region of the 16S rRNA gene (using primers described in [39]; TIB MOLBIOL Syntheselabor GmbH) directly form the bulk sample with a prolonged initial heating step of 5 min. After the amplicon PCR the genomic DNA was removed by AmPure XP Beads (Beckman Coulter Life Science Cat. No. A63881) with a 1:1.25 ratio of sample to beads (v/v). Next the amplicons were checked for their size and purity on a 1.5% agarose gel, and if suitable, subjected to the index PCR using the Nextera XT Index Kit v2 Set C (Illumina, FC-131-2003). After Index-PCR, the samples were cleaned again with AmPure XP Beads (Beckman Coulter Life Science Cat. No. A63881) in a 1:0.8 ratio of sample to beads (v/v). Samples were analysed by capillary gel electrophoresis (Agilent Fragment Analyser 5200) for correct size and purity with the NGS standard sensitivity fragment analysis kit (Agilent Cat. No. DF-473). Of all suitable samples a pool of 2 μM was generated and loaded to the Illumina MiSeq 2500 system.

### Sequence Alignment

Paired-end reads generated by Illumina MiSeq 16S rDNA sequencing were filtered and trimmed using Trimmomactic (Version 0.39) [40]. 7 leading bases with qualities below 35 were trimmed and reads shorter than 180 bases were filtered out. Using the DADA 2 (Version 1.22.0) software package [41], forward and reverse reads were truncated at 260 and 210 bases respectively and filtered with a minimum quality score of 12 and a maximum of 0 ambiguous nucleotides. Amplicon sequence variants (ASVs) were identified using the default settings of the DADA2 algorithm and ASVs were classified using the Silva 138.1 prokaryotic SSU taxonomic training data formatted for DADA 2[42]. After alignment of the sequences with DECIPHER (Version 2.24.0) [43], a phylogenetic tree was computed using FastTree (Version 2.1.11) [44].

### Graphical and Statistical Data Analysis

Statistical analyses were implemented through R (v. 4.0.3 or later versions), unless additionally stated. Computation of a-diversity index (Shannon index), and β-diversity with Bray-Curtis dissimilarity followed by Adonis test was conducted using vegan package. Graphical representation of differences between groups by Principal Coordinates Analysis (ggplot2, PCoA). Correlation analyses between mMFC and 16S rRNA sequencing was evaluated by Pearson's r using ggpubr package.

### Data Processing for Microbiota Phenotype Generation and Classification

Prior to any data processing the cohort comprising 103 IBD patients and 66 controls) was randomly split into a training and a hold-out validation set in the proportion of 70:30, wherein equivalent proportion of the number of samples of each group was maintained. The training set was used for data segmentation and modelling including the respective feature selection. The data segmentation map as well as the thereof selected features (discriminatory bins for classification) were applied to the validation set without re-computing, i.e. validation set was not included in the generation of microbiota phenotype nor model development.

The same strategy was for the sub-classification of IBD samples into CD and UC including all samples that were correctly classified as IBD dependent on the model (mMFC or 16 S rRNA). Here the cohort was again split randomly at a ratio of 70/30 for training and validation, maintaining the initial distribution of CD and UC samples. Data segmentation was maintained from the initial step, but the selection of discriminatory features (bins) was recomputed for the discrimination of CD and UC. The selected set of features was then applied to the validation set.

### Segmentation of Flow Cytometric Data

Raw FCS-files from flow cytometry were imported to R. Files were automatically gated for (1) scatter properties (FSC, SSC) to reduce instrument noise and (2) the DNA profile (Hoechst 33342, FSC) to exclude debris (Supp. FIG. 1). 10,000 events from each gated dataset were combined to segment the data by a self-organizing map (SOM, [24]). 2025 bins were used to describe the data comprising approx. 0.05% of events per bin. The optimal SOM model was mapped to each gated sample and combined to count tables. SOM procedures implemented in this study were conducted using kohonen, MASS, openCyto, flowWorkspace, and ggcyto [45-50] packages.

### Random Forest Models

Construction of a random forest machine learning model was used to classify samples [51, 52]. Briefly, (1) to reduce the number of features in both mMFC bins and 16S rRNA sequencing-derived genera table, all data were pre-filtered with the exclusion of non-significant features with p<0.05, Wilcoxon rank-sum test, followed by Bonferri-Holm p-value correction for multiple testing (vegan package). (2) For feature selection, Recursive Feature Elimination (RFE) with 10-fold cross-validation was applied to remove weak features for classification. The importance of the selected features was obtained within the RFE according to the consensus ranking through the 10-fold cross-validation. Evaluative parameters of the top 15 features are externally (SIAMCAT) determined on the entire data set without cross-validation for representation. The generalized fold change represents a more robust calculation for the large range of values, often including zero [53]. (3) The training set was used to train the random forest model involving 10 times repeated 10-fold cross-validation, to mitigate overfitting. Subsequently, the hold-out validation set was used to evaluate the models' predictive performance in one modelling attempt. All procedures related to model construction were performed in caret package [25], and evaluation of predictive ability (performance metrics, AUROC, sensitivity, specificity, and confusion matrix at default threshold 0.5) was implemented using MLeval package [54]. Relative abundances of bacterial genera identified in sequenced samples and normalized mMFC cluster counts were used as inputs while building the model.

### REFERENCES

1. Clemente, J.C., et al., The impact of the gut microbiota on human health: an integrative view. Cell, 2012. 148(6): p. 1258-70.

2. Fan, Y. and O. Pedersen, Gut microbiota in human metabolic health and disease. Nat Rev Microbiol, 2021. 19(1): p. 55-71.

3. Morais, L.H., H.L.t. Schreiber, and S.K. Mazmanian, The gut microbiota-brain axis in behaviour and brain disorders. Nat Rev Microbiol, 2021. 19(4): p. 241-255.

4. Cheng, W.Y., C.Y. Wu, and J. Yu, The role of gut microbiota in cancer treatment: friend or foe? Gut, 2020. 69(10): p. 1867-1876.

5. Duvallet, C., et al., Meta-analysis of gut microbiome studies identifies disease-specific and shared responses. Nat Commun, 2017. 8(1): p. 1784.

6. Walters, W.A., Z. Xu, and R. Knight, Meta-analyses of human gut microbes associated with obesity and IBD. FEBS Lett, 2014. 588(22): p. 4223-33.

7. Durack, J. and S.V. Lynch, The gut microbiome: Relationships with disease and opportunities for therapy. J Exp Med, 2019. 216(1): p. 20-40.

8. Metwaly, A., S. Reitmeier, and D. Haller, Microbiome risk profiles as biomarkers for inflammatory and metabolic disorders. Nat Rev Gastroenterol Hepatol, 2022. 19(6): p. 383-397.

9. Knox, N.C., et al., The Gut Microbiome as a Target for IBD Treatment: Are The inventors There Yet? Curr Treat Options Gastroenterol, 2019. 17(1): p. 115-126.

10. Human Microbiome Project, C., Structure, function and diversity of the healthy human microbiome. Nature, 2012. 486(7402): p. 207-14.

11. Lloyd-Price, J., et al., Multi-omics of the gut microbial ecosystem in inflammatory bowel diseases. Nature, 2019. 569(7758): p. 655-662.

12. Serrano-Gomez, G., et al., Dysbiosis and relapse-related microbiome in inflammatory bowel disease: A shotgun metagenomic approach. Comput Struct Biotechnol J, 2021. 19: p. 6481-6489.

13. Palm, N.W., et al., Immunoglobulin A coating identifies colitogenic bacteria in inflammatory bowel disease. Cell, 2014. 158(5): p. 1000-1010.

14. Frehn, L., et al., Distinct patterns of IgG and IgA against food and microbial antigens in serum and feces of patients with inflammatory bowel diseases. PLOS One, 2014. 9(9): p. e106750.

15. Masu, Y., et al., Immunoglobulin subtype-coated bacteria are correlated with the disease activity of inflammatory bowel disease. Sci Rep, 2021. 11(1): p. 16672.

16. Koch, C., et al., Cytometric fingerprinting for analyzing microbial intracommunity structure variation and identifying subcommunity function. Nat Protoc, 2013. 8(1): p. 190-202.

17. Esser, C., et al., Beyond sequencing: fast and easy microbiome profiling by flow cytometry. Arch Toxicol, 2019. 93(9): p. 2703-2704.

18. Ruben Props, P.M., Mohamed Mysara, Lieven Clement, Nico Boon, Measuring the biodiversity of microbial communities by flow cytometry. 2016. 7(11).

19. Zimmermann, J., et al., High-resolution microbiota flow cytometry reveals dynamic colitis-associated changes in fecal bacterial composition. Eur J Immunol, 2016. 46(5): p. 1300-3.

20. Schmiester, M., et al., Flow cytometry can reliably capture gut microbial composition in healthy adults as well as dysbiosis dynamics in patients with aggressive B-cell non-Hodgkin lymphoma. Gut Microbes, 2022. 14(1): p. 2081475.

21. Krause, J.L., et al., The Activation of Mucosal-Associated Invariant T (MAIT) Cells Is Affected by Microbial Diversity and Riboflavin Utilization in vitro. Front Microbiol, 2020. 11: p. 755.

22. Kupschus, J., et al., Rapid detection and online analysis of microbial changes through flow cytometry. Cytometry A, 2022.

23. Rubbens, P., et al., Cytometric fingerprints of gut microbiota predict Crohn's disease state. ISME J, 2021. 15(1): p. 354-358.

24. Kohonen, T., Self-Organizing Maps. 3 ed. Springer Series in Information Sciences. 2001: Springer Berlin, Heidelberg.

25. Kuhn, M., caret: Classification and Regression Training. 2016.

26. Linares-Blanco, J., et al., Machine Learning Based Microbiome Signature to Predict Inflammatory Bowel Disease Subtypes. Front Microbiol, 2022. 13: p. 872671.

27. Kang, X., et al., Reprocessing 16S rRNA Gene Amplicon Sequencing Studies: (Meta)Data Issues, Robustness, and Reproducibility. Front Cell Infect Microbiol, 2021. 11: p. 720637.

28. Clooney, A.G., et al., Ranking microbiome variance in inflammatory bowel disease: a large longitudinal intercontinental study. Gut, 2021. 70(3): p. 499-510.

29. Sarrabayrouse, G., et al., Fungal and Bacterial Loads: Noninvasive Inflammatory Bowel Disease Biomarkers for the Clinical Setting. mSystems, 2021. 6(2).

30. Michaud, E., et al., Alteration of microbiota antibody-mediated immune selection contributes to dysbiosis in inflammatory bowel diseases. EMBO Mol Med, 2022: p. e15386.

31. Bourgonje, A.R., et al., Patients With Inflammatory Bowel Disease Show IgG Immune Responses Towards Specific Intestinal Bacterial Genera. Front Immunol, 2022. 13: p. 842911.

32. Tytgat, H.L.P. and W.M. de Vos, Sugar Coating the Envelope: Glycoconjugates for Microbe-Host Crosstalk. Trends Microbiol, 2016. 24(11): p. 853-861.

33. Franzosa, E.A., et al., Gut microbiome structure and metabolic activity in inflammatory bowel disease. Nat Microbiol, 2019. 4(2): p. 293-305.

34. Matute, J.D., et al., Intelectin-1 binds and alters the localization of the mucus barrier-modifying bacterium Akkermansia muciniphila. J Exp Med, 2023. 220(1).

35. Gupta, V.K., et al., A predictive index for health status using species-level gut microbiome profiling. Nat Commun, 2020. 11(1): p. 4635.

36. Martinez-Guryn, K., V. Leone, and E.B. Chang, Regional Diversity of the Gastrointestinal Microbiome. Cell Host Microbe, 2019. 26(3): p. 314-324.

37. Ninnemann J., B.L., Bondareva M., Witkowski M., Angermair S., Induction of cross-reactive antibody responses against the RBD domain of the spike protein of SARS-COV-2 by commensal microbiota. BioRxiv, 2021.

38. Bellais, S., et al., Species-targeted sorting and cultivation of commensal bacteria from the gut microbiome using flow cytometry under anaerobic conditions. Microbiome, 2022. 10(1): p. 24.

39. Klindworth, A., et al., Evaluation of general 16S ribosomal RNA gene PCR primers for classical and next-generation sequencing-based diversity studies. Nucleic Acids Res, 2013. 41(1): p. e1.

40. Bolger, A.M., M. Lohse, and B. Usadel, Trimmomatic: a flexible trimmer for Illumina sequence data. Bioinformatics, 2014. 30(15): p. 2114-20.

41. Callahan, B.J., et al., DADA 2: High-resolution sample inference from Illumina amplicon data. Nat Methods, 2016. 13(7): p. 581-3.

42. McLaren, M.R.C., B. J., Silva 138.1 prokaryotic SSU taxonomic training data formatted for DADA2. Zenodo, 2021.

43. ES, W., Using DECIPHER v2.0 to analyze big biological sequence data in R. The R Journal, 2016. 8(1): p. 352-359.

44. Price, M.N., P.S. Dehal, and A.P. Arkin, FastTree: computing large minimum evolution trees with profiles instead of a distance matrix. Mol Biol Evol, 2009. 26(7): p. 1641-50.

45. Wehrens R., B.L.M.C., Self- and Super-organizing Maps in R: The kohonen Package. Journal of Statistical Software, 2007. 21(5).

46. Wehrens R., K.J., Flexible Self-Organizing Maps in kohonen 3.0. Journal of Statistical Software, 2018. 87(7).

47. Van, P., et al., ggCyto: next generation open-source visualization software for cytometry. Bioinformatics, 2018. 34(22): p. 3951-3953.

48. Ripley, W.N.V.a.B.D., Modern Applied Statistics with S. 4 ed. 2002: Springer, New York.

49. Finak, G., et al., OpenCyto: an open source infrastructure for scalable, robust, reproducible, and automated, end-to-end flow cytometry data analysis. PLOS Comput Biol, 2014. 10(8): p. e1003806.

50. Finak G, J.M., flowWorkspace: Infrastructure for representing and interacting with gated and ungated cytometry data sets. 2022.

51. Ye, L., et al., Machine learning-aided analyses of thousands of draft genomes reveal specific features of activated sludge processes. Microbiome, 2020. 8(1): p. 16.

52. Jacobs, J.P., et al., Cognitive behavioral therapy for irritable bowel syndrome induces bidirectional alterations in the brain-gut-microbiome axis associated with gastrointestinal symptom improvement. Microbiome, 2021. 9(1): p. 236.

53. Wirbel, J., et al., Meta-analysis of fecal metagenomes reveals global microbial signatures that are specific for colorectal cancer. Nat Med, 2019. 25(4): p. 679-689.

54. John, C.R., MLeval: Machine Learning Model Evaluation. 2020.

55. Özel Duygan Birge D et al., Recent advances in microbial community analysis from machine learning of multiparametric flow cytometry data, CURRENT OPINION IN BIOTECHNOLOGY, LONDON, GB, vol. 75, 2 Feb. 2022.

56. Rubbens Peter et al., Cytometric fingerprints of gut microbiota predict Crohn's disease state, THE ISME JOURNAL, vol. 15, no. 1, pages 354-358.

57. Jackson Matthew A. et al., Accurate identification and quantification of commensal microbiota bound by host immunoglobulins, Microbiome, vol. 9, no. 1, 30 Jan. 2021.

58. Bourgonje Arno R et al., Antibody signatures in inflammatory bowel disease: current developments and future applications, TRENDS IN MOLECULAR MEDICINE, ELSEVIER CURRENT TRENDS GB, vol. 28, no. 8, 31 May 2022, pages 693-705.

1. A method for identifying a multi-parameter phenotype of microbiota, comprising:
(i) providing a sample comprising microbiota,
(ii) labeling said microbiota with multiple labels, each of which binds a phenotypic parameter of said microbiota,
(iii) detecting an intensity of the labelled phenotypic parameters of single cells of the microbiota by flow cytometry, and
(iv) segmenting the single cells into bins based on the intensities of detected phenotypic parameters, wherein the distribution of single cells in bins represents a multi-parameter phenotype of said microbiota.

2. The method according to claim 1, further comprising:
(v) analyzing the binned cells that define a multi-parameter phenotype using a predetermined model comprising one or more established patterns that represent signature(s) associated with a state, and/or
(vi) analyzing the binned cells using a machine learning model trained on correlation(s) between two or more states.

3. The method according to claim 2, wherein the state is a medical condition in a subject.

4. The method according to claim 1, wherein the phenotypic parameter of the microbiota labelled in step ii) is a structural feature of a microorganism and/or human immunoglobulin bound to the microorganism.

5. The method according to claim 4, wherein:
(a) the binding agent that specifically recognizes a host immunoglobulin is an antibody or antigen binding fragment thereof that binds an immunoglobulin isotype IgA1, IgA2, IgG, IgD, IgE and/or IgM, and/or
(b) wherein the binding agent that specifically recognizes a sugar molecule binds a sugar present on the surface of a cell of the microbiota.

6. The method according to claim 1, wherein step ii) comprises additionally using a label that binds a nucleic acid molecule in single cells of the microbiota, and selecting cells with a positive nucleic acid molecule signal prior to grouping in step iv).

7. The method according to claim 1, wherein each bin is defined by at least one intensity value of a label binding to a host immunoglobulin and at least one intensity value of a label binding to a sugar molecule.

8. The method according to claim 1, further comprising selecting one or more discriminatory bins for distinguishing between two inflammatory bowel diseases.

9. The method according to claim 1, further comprising generating a pattern that represents a signature for an inflammatory bowel disease based on the distribution of single cells into bins based on the intensities of detected phenotypic parameters, or based on the discriminatory bins according to claim 8.

10. The method according to claim 1, wherein the inflammatory bowel disease is Crohn's disease or Ulcerative colitis.

11. The method according to claim 1, wherein the sample is a stool sample or other sample comprising microbiota, saliva.

12. The method according to claim 1, further comprising training a machine-learning model on one or more correlations between a multi-parameter phenotype of said microbiota and one or more states in a subject.

13. A system for identifying a multi-parameter phenotype of intestinal microbiota, comprising
(a) a flow cytometer,
(b) a data store comprising one or more reference values, intensity values and/or data defining one or more bins based on phenotypic parameters of single cells of the microbiota,
(c) a software configured for analyzing the intensities of labelled phenotypic parameters of single cells of the microbiota, using data generated by flow cytometry, and segmenting the single cells into bins based on the intensities of detected phenotypic parameters,
(d) wherein the software is configured for identifying a medical condition, such as an inflammatory bowel disease, associated with a signature for said condition, based on the grouping of single cells into bins based on the intensities of detected phenotypic parameters.

14. A kit for identifying a multi-parameter phenotype of microbiota, comprising
(a) multiple labels, each for binding a phenotypic parameter of microbiota, and
(b) a software configured for analyzing the intensities of labelled phenotypic parameters of single cells of the microbiota, using data generated by flow cytometry, and segmenting the single cells into bins based on the intensities of detected phenotypic parameters,
(c) wherein said software contains a reference profile for at least one state.

15. A method for diagnosing a medical condition associated with microbiota, in a subject, comprising:
(i) identifying a multi-parameter phenotype of intestinal microbiota of a subject with a method according to claim 1, and
(ii) evaluating the phenotype using a machine-learning model that is trained on one or more correlations between a multi-parameter phenotype of said microbiota and one or more states in the subject.

16. The method according to claim 1, wherein the label in step ii) comprises a binding agent that specifically recognizes a host immunoglobulin or a sugar molecule present on the surface of a cell of the microbiota.

17. The method of claim 3, wherein the medical condition is a chronic inflammatory disease or inflammatory bowel disease

18. The method according to claim 5, wherein the sugar present on the surface of a cell of the microbiota is a lactose-, mannose- and/or N-Acetyl-glucosamine-structure.

19. The method according to claim 18, wherein the label is a plant-derived lectin, wherein the plant-derived lectin comprises one or more of peanut agglutinin (PNA), Concanavalin A (ConA), Solanum tuberosum agglutinin (STA) and wheat germ agglutinin (WGA).

20. The method of claim 11, wherein the sample is saliva, a nasopharyngeal swab or a skin swab.

21. The kit for identifying a multi-parameter phenotype of microbiota according to claim 14, wherein the software in contains a reference profile for two states.

22. The method for diagnosing a medical condition associated with microbiota according to claim 15, wherein said medical condition is an inflammatory bowel disease, wherein said inflammatory bowel disease is Crohn's disease and/or ulcerative colitis.
